SEQUENCE LISTING (38 sequences)

SEQ ID NO: 1 - DNA, 1984 bp, Brassica napus
gagtcatcatcgtgaaacatttattgattacaaaacaaaaaaaaagaaaatatttaccaaggaacaatggtgtgagaggttttcgtctccgcgcagaagatttttatttatttaaattacggttttttttctgagtagaattatactactatggagtgtgttggtgctcgcaatctcgctgcaacggcggtcacagattttccgtcctggagttcttcgcgtaaaaactatcccgtggttaatagatacagctttagtaatctccggtgcggtttgtgtagagtcaaagctagcggcggcggagcaggttccggtatagagagttgcgtggcggtgagagaggacttcgccgacaaggaagacttcgtgaaggctggtggttcggagattctatacgttcaaatgcagcagaacaaagacatggatgaacagtctaagcttgttgataagttacctcctatatcaactggtgaaggtggtggtgctttggacctagtggttattgggtgtggtcctgctggtttagccttggcggctgaatcagctaagttaggacttaaagttggactgattggtcctgaccttcctttcactaacaactacggtgtttgggaagatgagttcaacgatcttggcttgcaaaaatgtattgagcatgtttggagagatacccttgtgtatctggacgatgacaatcctattaccattggtcgtgcttatggaagagttagtcgacgtttacttcacgaggagttcttgaggaggtgtgtggagtcaggtgtctcgtatcttagctccaaagttgagagcataacagaagctcctgatggccttaggcttgtttcctgtgaacaaaacacccttgttccgtgcaggcttgccactgttgcttctggagcagcttctgggaagctcttgcaatacgaagttggagggcctagagtctgtgtccaaattgcttacggcttggaggttgaggtggaaaagagtccatatgatccagagcagatggtgttcatggattacagagattatacaaacgagaaaatccggagcttagaagctgaatatccagcgtttctctacgccatgcctatgacaaagaccagagtcttcttcgaggagacatgtcttgcttcaaaagatgtcatgccctttgatttgcttaaaaagaagctcttgttgagattagagacactcggaatccgaatactaaagacttacgaagaggaatggtcttatatcccagtaggtggttccttgccaaacacggaacaaaagaatctcgcctttggtgctgcagctagcatggttcatcctgcaacaggctattcagttgtgagatctttgtctgaagctccaaaatacgcatcagtcatcgctaatatactaaaacatgagaccactacttccttcaccagacacatcaacaccaatatttcaagacaagcttgggatactttatggccaccagaaaggaaacgacaaagagcattctttctctttggccttgcgctcatagttcaactcgacatcgaaggcattagatgcttcttccacactttcttccgccttccaaaatggatgtggagagggtttctaggatcaacattaacatcaggagacctcgttctgtttgctttctacatgttcatcattgcaccaaacaacttgagaaaaggtctcatcaatcatcttatctctgatccaaccggagcaaccatgattaaaacctatcttaaagtatgattctctcctccactcttgggtttttatatatatgaataatccatcgacgaatggcatgtgggttactaggaaaatttgtaacaaacgtttatagaatctattattcacaaaggtgcgtgccttagaagattgagagaaggatagtctttgtggattttagaattgccattttatttaaatacgatccattctcgttgttacattaaaaaaaaaaaaaaaaaaaaaaaaaaaaaaa

SEQ ID NO: 2 - DNA, 384 bp, Brassica napus
cgactagtggcgcgccgaggttttcgtctccgcgcagaagatttttatttatttaaattacggttttttttctgagtagaattatactactatggagtgtgttggtgctcgcaatctcgctgcaacggcggtcacagattttccgtcctggagttcttcgcgtaaaaactatcccgtggttaatagatacagctttagtaatctccggtgcggtttgtgtagagtcaaagctagcggcggcggagcaggttccggtatagagagttgcgtggcggtgagagaggacttcgccgacaaggaagacttcgtgaaggctggtggttcggagattctatacgttcaaatgcagcagaacaaagacatggatgatttaaatggatcccg

SEQ ID NO: 3 - DNA, 442 bp, Brassica napus
cgactagtggcgcgcccagaaaggaaacgacaaagagcattctttctctttggccttgcgctcatagttcaactcgacatcgaaggcattagatgcttcttccacactttcttccgccttccaaaatggatgtggagagggtttctaggatcaacattaacatcaggagacctcgttctgtttgctttctacatgttcatcattgcaccaaacaacttgagaaaaggtctcatcaatcatcttatctctgatccaaccggagcaaccatgattaaaacctatcttaaagtatgattctctcctccactcttgggtttttatatatatgaataatccatcgacgaatggcatgtgggttactaggaaaatttgtaacaaacgtttatagaatctattattcacaaaggtgcgtgccttagaagattgatttaaatggatcccg

SEQ ID NO: 4 - DNA, 1901 bp, Arabidopsis thaliana
acaaaaggaaataattagattcctctttctgcttgctataccttgatagaacaatataacaatggtgtaagtcttctcgctgtattcgaaattatttggaggaggaaaatggagtgtgttggggctaggaatttcgcagcaatggcggtttcaacatttccgtcatggagttgtcgaaggaaatttccagtggttaagagatacagctataggaatattcgtttcggtttgtgtagtgtcagagctagcggcggcggaagttccggtagtgagagttgtgtagcggtgagagaagatttcgctgacgaagaagattttgtgaaagctggtggttctgagattctatttgttcaaatgcagcagaacaaagatatggatgaacagtctaagcttgttgataagttgcctcctatatcaattggtgatggtgctttggatctagtggttattggttgtggtcctgctggtttagccttggctgcagaatcagctaagcttggattaaaagttggactcattggtccagatcttccttttactaacaattacggtgtttgggaagatgaattcaatgatcttgggctgcaaaaatgtattgagcatgtttggagagagactattgtgtatctggatgatgacaagcctattaccattggccgtgcttatggaagagttagtcgacgtttgctccatgaggagcttttgaggaggtgtgtcgagtcaggtgtctcgtaccttagctcgaaagttgacagcataacagaagcttctgatggccttagacttgttgcttgtgacgacaataacgtcattccctgcaggcttgccactgttgcttctggagcagcttcgggaaagctcttgcaatacgaagttggtggacctagagtctgtgtgcaaactgcatacggcgtggaggttgaggtggaaaatagtccatatgatccagatcaaatggttttcatggattacagagattatactaacgagaaagttcggagcttagaagctgagtatccaacgtttctgtacgccatgcctatgacaaagtcaagactcttcttcgaggagacatgtttggcctcaaaagatgtcatgccctttgatttgctaaaaacgaagctcatgttaagattagatacactcggaattcgaattctaaagacttacgaagaggagtggtcctatatcccagttggtggttccttgccaaacaccgaacaaaagaatctcgcctttggtgctgccgctagcatggtacatcccgcaacaggctattcagttgtgagatctttgtctgaagctccaaaatatgcatcagtcatcgcagagatactaagagaagagactaccaaacagatcaacagtaatatttcaagacaagcttgggatactttatggccaccagaaaggaaaagacagagagcattctttctctttggtcttgcactcatagttcaattcgataccgaaggcattagaagcttcttccgtactttcttccgccttccaaaatggatgtggcaagggtttctaggatcaacattaacatcaggagatctcgttctctttgctttatacatgttcgtcatttcaccaaacaatttgagaaaaggtctcatcaatcatctcatctctgatccaaccggagcaaccatgataaaaacctatctcaaagtatgatttacttatcaactcttaggtttgtgtatatatatgttgatttatctgaataatcgatcaaagaatggtatgtgggttactaggaagttggaaacaaacatgtatagaatctaaggagtgatcgaaatggagatggaaacgaaaagaaaaaaatcagtctttgttttgtggttagtgaccgagaaatgtttaaatcaacaagagtaaaaatgttcaac

SEQ ID NO: 5 - DNA, 32 bp, Artificial Sequence
  Synthetic primer
cgactagtggcgcgccgaggttttcgtctccg

SEQ ID NO: 6 - DNA, 35 bp, Artificial Sequence
  Synthetic primer
cgggatccatttaaatcatccatgtctttgttctg

SEQ ID NO: 7 - DNA, 33 bp, Artificial Sequence
  Synthetic primer
cgactagtggcgcgcccagaaaggaaacgacaa

SEQ ID NO: 8 - DNA, 34 bp, Artificial Sequence
  Synthetic primer
cgggatccatttaaatcaatcttctaaggcacgc

SEQ ID NO: 9 - DNA, 26 bp, Artificial Sequence
  Synthetic primer
caatcccactatccttcgcaagaccc

SEQ ID NO: 10 - DNA, 16 bp, Artificial Sequence
  Synthetic primer
gaggttttcgtctccg

SEQ ID NO: 11 - DNA, 19 bp, Artificial Sequence
  Synthetic primer
catccatgtctttgttctg

SEQ ID NO: 12 - DNA, 25 bp, Artificial Sequence
  Synthetic primer
cttagacaagcggctttggtgaaca

SEQ ID NO: 13 - DNA, 24 bp, Artificial Sequence
  Synthetic primer
actgagctcggtgacgcctttctc

SEQ ID NO: 14 - DNA, 25 bp, Artificial Sequence
  Synthetic primer
cgagatgctgacatggccagagaaa

SEQ ID NO: 15 - DNA, 25 bp, Artificial Sequence
  Synthetic primer
agtggcaaacacataagcgtctcct

SEQ ID NO: 16 - DNA, 24 bp, Artificial Sequence
  Synthetic primer
agagaggatggttgcgaggctgaa

SEQ ID NO: 17 - DNA, 24 bp, Artificial Sequence
  Synthetic primer
aacagcctcgacgacaagaaacca

SEQ ID NO: 18 - DNA, 26 bp, Artificial Sequence
  Synthetic primer
caccagaaaggaaacgacaaagagca

SEQ ID NO: 19 - DNA, 26 bp, Artificial Sequence
  Synthetic primer
ttctctcaatcttctaaggcacgcac

SEQ ID NO: 20 - DNA, 16 bp, Artificial Sequence
  Synthetic primer
gaggttttcgtctccg

SEQ ID NO: 21 - DNA, 26 bp, Artificial Sequence
  Synthetic primer
ttctctcaatcttctaaggcacgcac

SEQ ID NO: 22 - DNA, 23 bp, Artificial Sequence
  Synthetic primer
tgaaagatggccgatggtgagga

SEQ ID NO: 23 - DNA, 24 bp, Artificial Sequence
  Synthetic primer
ccgtctccagctcttgctcgtagt

SEQ ID NO: 24 - DNA, 23 bp, Artificial Sequence
  Synthetic primer
tgaaagatggccgatggtgagga

SEQ ID NO: 25 - DNA, 25 bp, Artificial Sequence
  Synthetic primer
cacactcaccaccacgaaccagaag

SEQ ID NO: 26 - DNA, 33 bp, Artificial Sequence
  Synthetic primer
gcggatccaatgctagcttcaatggcagcggca

SEQ ID NO: 27 - DNA, 37 bp, Artificial Sequence
  Synthetic primer
gcgagctcctataaggcattcatacgctttattcttc

SEQ ID NO: 28 - DNA, 1964 bp, Arabidopsis thaliana
ctctctttctgggaagaagataattgtctccatctccatgaagctactgcttctgggtaagttttgtggtcttcgttcatcttttctctagcaatttagtattccattttctcaatccctctggttagaaatcgtgtccggtgatttttgaattatatccttttggtgttttcttcgattttgggggaattcgatggatactctgttgaaaacacccaacaagctcgattttttcatccctcagtttcatgggtttgagagattatgcagtaacaatccataccattcaagggttaggcttggtgtgaagaaaagggctatcaaaattgtctctagtgtagtgagtggtagcgctgctcttttggatcttgttcctgaaactaagaaggagaatcttgactttgagcttcctttgtacgacacttccaagagtcaagttgttgatttggctattgttggtggtggtcctgctggtttagccgtggctcagcaggtttctgaagctggactctctgtttgttccattgatccttctcctaagctcatatggcctaacaattatggagtttgggttgatgagtttgaggctatggatttactagactgcctggataccacatggtctggtgctgttgtctatgtcgatgaaggtgtcaagaaggatttgagccggccttatgggagagttaaccggaaacagctcaaatccaaaatgcttcagaaatgtattaccaacggtgttaaatttcatcagtctaaggtcactaatgtggttcacgaggaggcaaactccactgtggtctgcagtgacggtgtaaagattcaggcttccgtggttcttgatgccactgggttttcccgatgcttggttcagtatgacaaaccttacaaccctgggtaccaagtagcttacgggattgtagctgaagttgatggtcacccattcgatgtagacaaaatggtgttcatggattggagagacaaacatctggactcatatcctgagctgaaagaacggaacagcaagatcccaacgttcttgtacgctatgccattttcttccaaccgaatatttcttgaagaaacttctttagttgctagacctggtctgagaatggaagatatccaagaaagaatggctgctagactgaaacatctggggatcaatgtgaagaggattgaggaagacgagcgttgtgtgatcccgatgggcggtcctttaccagtcttacctcaacgggttgtggggattggtgggacagcaggaatggttcatccttcaactggttacatggttgctaggactcttgcagctgcaccaatagttgcaaatgccattgtgagatacctcggttcaccaagtagtaatagcctgagaggagatcaactctctgctgaggtttggagagacttgtggcctatcgaacggcgtagacagagggagttcttctgttttggaatggatattctgctgaaactcgatttagacgctactagaaggttctttgatgcattctttgatctgcaacctcattactggcacggattcttgtcttccaggctgtttctcccggaactgttggtcttcgggttgtcgctcttctcacacgcttccaatacctcaagattggagatcatgacaaaggggactgttcctcttgctaagatgatcaacaatttggtacaagatagagactaaggaccagaaacttagacatataagtacatctgttctttggttcttgaccagtagtatatccgcattgcaagtcgttggataattgtgtataaaccacagatcataacctgaatccttgtgaaatcaaattgttactactagttcattaaaattaatactttgtgctgcattgtgtttcaccaactcttgtaaatccaaaactagaggcaaaatgtaataagataaaagagtggattttgaacacaaaagcaaatctttcaaactc

SEQ ID NO: 29 - DNA, 34 bp, Artificial Sequence
  Synthetic primer
gctctagagatggcagcagcaatttcagtgttca

SEQ ID NO: 30 - DNA, 35 bp, Artificial Sequence
  Synthetic primer
gcgagctctcaggtagatggttgcgttcgtttagt

SEQ ID NO: 31 - DNA, 231 bp, Brassica napus
tctagatgcatgctcgagcggccgccagtgtgatggatatctgcagaattcgatggatactctgttgaaaacacccaacaagctcgagtttttcatccctcagtttcatgggttcgagagattaagtccaaatccttcaagggttaagcttggtttgaagaaaagagcaatcagaatcggtagtagtatcactagtgctagtagtgctcttttggatcttgttcctgaaac

SEQ ID NO: 32 - DNA, 320 bp, Brassica napus
attgagaggcggagacagagggagttcttctgttttgggatggatattttgctgaagcttgatttggatgctactaggaggttctttgatgcgttctttgacctggaaccgcgttactggcatggtttcttgtcgtcgaggctgtttctcccggacctggtgttcttcgggctgtcgctcttctcgcatgcttctaatacctcgaggttggagatcatgacgaagggaactgttcctcttgctaagatgatcaacaatttggtacaagatagaagccgaattccagcacactggcggccgttactagtggatccgagctc

SEQ ID NO: 33 - DNA, 1039 bp, Brassica napus
caagaaggagaatctcgacttggaccttcctttaaacgacacttccctgaacaaagttgttgatctagctatcgttggcggcggccccgctggtttagccgtggctcagcaagtctccgaagctggactctctgtctgctccatcgatccttcccccaaactcatttggcctaacaactacggagtttgggttgacgagttcgaagccatggacttgctagactgcctcgacaccacctggtccggcgccgtcgtctacatcaacgacggctccgaaaaggacctgtcccggccttacgggagagtgaaccgtaaacagctcaaatccaagatgcttcagaagtgcatcaccaacggtgttagattccatcaggctaaagtcactgacgtggttcacgaggaggttaactccactgttgtctgcagtgacggtgtgaagattcaggcttctgttgttctcgacgctactggtttttcaagatgcttggttcagtatgataagccttataaccctgggtatcaagtagcttatggtatcattgctgaggtcgatggtcacccgtttgatgtggataagatggtgttcatggactggagagataagcatcttgacgcgtaccctgaggttaaagagcggaacagcaagatccctacgttcctgtacgcgatgccgttttcttccaacaggatctttcttgaagagacgtctcttgtggctaggccgggtctgaagatggaagatatccaagagaggatggttgcgaggctgaaacatttggggatcaacgtgaagcggattgaggaagacgagcgttgtgtgatccctatgggaggtcctttaccggtcttgcctcaaagagttgttggcattggcggtacggcggggatggttcatccttcgactggttacatggttgctaggactcttgcagctgctccgatagttgcaaacgctatagtgcggtaccttggtggtggtaacaacaacggcttgagaggagatgagctctcggctgaggtgtggagagacttgtggcct

SEQ ID NO: 34 - DNA, 1042 bp, Brassica napus
acagtctaagcttgttgataagttacctcctatatcaactggtgaaggtggtggtgctttggacctagtggttattgggtgtggtcctgctggtttagccttggcggctgaatcagctaagttaggacttaaagttggactgattggtcctgaccttcctttcactaacaactacggtgtttgggaagatgagttcaacgatcttggcttgcaaaaatgtattgagcatgtttggagagatacccttgtgtatctggacgatgacaatcctattaccattggtcgtgcttatggaagagttagtcgacgtttacttcacgaggagttcttgaggaggtgtgtggagtcaggtgtctcgtatcttagctccaaagttgagagcataacagaagctcctgatggccttaggcttgtttcctgtgaacaaaacacccttgttccgtgcaggcttgccactgttgcttctggagcagcttctgggaagctcttgcaatacgaagttggagggcctagagtctgtgtccaaattgcttacggcttggaggttgaggtggaaaagagtccatatgatccagagcagatggtgttcatggattacagagattatacaaacgagaaaatccggagcttagaagctgaatatccagcgtttctctacgccatgcctatgacaaagaccagagtcttcttcgaggagacatgtcttgcttcaaaagatgtcatgccctttgatttgcttaaaaagaagctcttgttgagattagagacactcggaatccgaatactaaagacttacgaagaggaatggtcttatatcccagtaggtggttccttgccaaacacggaacaaaagaatctcgcctttggtgctgcagctagcatggttcatcctgcaacaggctattcagttgtgagatctttgtctgaagctccaaaatacgcatcagtcatcgctaatatactaaaacatgagaccactacttccttcaccagacacatcaacaccaatatttcaagacaagcttgggatactttatggcca

SEQ ID NO: 35 - DNA, 400 bp, Brassica napus
gagtcatcatcgtgaaacatttattgattacaaaacaaaaaaaaagaaaatatttaccaaggaacaatggtgtgagaggttttcgtctccgcgcagaagatttttatttatttaaattacggttttttttctgagtagaattatactactatggagtgtgttggtgctcgcaatctcgctgcaacggcggtcacagattttccgtcctggagttcttcgcgtaaaaactatcccgtggttaatagatacagctttagtaatctccggtgcggtttgtgtagagtcaaagctagcggcggcggagcaggttccggtatagagagttgcgtggcggtgagagaggacttcgccgacaaggaagacttcgtgaaggctggtggttcggagattctatacgttc

SEQ ID NO: 36 - DNA, 514 bp, Brassica napus
ccagaaaggaaacgacaaagagcattctttctctttggccttgcgctcatagttcaactcgacatcgaaggcattagatgcttcttccacactttcttccgccttccaaaatggatgtggagagggtttctaggatcaacattaacatcaggagacctcgttctgtttgctttctacatgttcatcattgcaccaaacaacttgagaaaaggtctcatcaatcatcttatctctgatccaaccggagcaaccatgattaaaacctatcttaaagtatgattctctcctccactcttgggtttttatatatatgaataatccatcgacgaatggcatgtgggttactaggaaaatttgtaacaaacgtttatagaatctattattcacaaaggtgcgtgccttagaagattgagagaaggatagtctttgtggattttagaattgccattttatttaaatacgatccattctcgttgttacattaaaaaaaaaaaaaaaaaaaaaaaaaaaaaaa

SEQ ID NO: 37 - DNA, 1187 bp, Adonis aestivalis
cagtactcggggtcttccaaaaacctccaacaaagctcatactttctatctcgaatgctagcttcaatggcagcggcaacctcaatcacttcaagctccagagcatttcgtttccacagaggtctgttcttaaacacaaaacccaacattcgtaatcccccttgtttgctattttctccacttctgatgagaaacagaaacggtgctggtgctttgactatctgctttgttgcagagagaacaagggggcgtgagattccccagattgaagaggacgaaaaaaacatggatgaagtgtttgagcagatgaattcagcttcagtacgagttgccgagaagttagctagaaagaaatcagaaaggtttacttatctcattgctgctctcatgtctagtatgggcatcacttctatggctattctttctgtttattacagattttcttggcaaatggaggggggagatattcctgttacagaaatgttgggcacatttgctctatctgttggtgctgctgtaggaatggagttttgggcaagatgggctcacagagcactctggcatgcttctctatggcacatgcacgagtcccaccataagccaagagaaggccctttcgagctaaatgacgtcttcgccattattaacgcggttccagctattgctcttctcaattttggcttcttccacaaaggcctcattcctggtctatgctttggtgctggtcttggaattacagtatttggcatggcttatatgtttgtccacgacggcctagttcatcgaagattcccagtgggaccaattgcaaatgttccttatttccgcaaagtggctgctgctcaccaaattcatcacacagacaaattccaaggtgtaccatatggattgtttcttggaccaaaggaattagaagaggtaggaggaaatgaagagttggagaaggagatagagcgaagaataaagcgtatgaatgccttatagtcacaggtagtcggagaggtcattcacttaccaaaatgtgataacgattttgatacatcagtggagaggaagcgcagaaagtcttgtgttgtattatacattgtatcaatagataagatgaaagcagaaaagagtcgtactcacaacgtgtacatgtgtttattaatgttagataatctattttcgtgcataaaaaaaaaaaa

SEQ ID NO: 38 - DNA, 1120 bp, Adonis aestivalis
atggcagcagcaatttcagtgttcagttcaggttattctttctacaagaatctcttgttggactcaaaaccaaatattctcaaacccccatgcctgctattctctccagttgtgatcatgtcgcctatgagaaagaaaaagaaacatggtgatccatgtatctgctccgttgcagggagaacaaggaaccttgatattcctcaaattgaagaagaggaagagaatgtggaagaactaatagaacagaccgattctgacatagtgcatataaagaaaacactaggggggaaacaatcaaaacggcccactggctccattgtcgcacccgtatcttgtcttgggatcctttcaatgattggacctgctgtttacttcaagttttcacggctaatggagggtggagatatacctgtagcagaaatggggattacgtttgccacctttgttgctgctgctgttggcacggagtttttgtcagcatgggttcacaaagaactctggcacgagtctttgtggtacattcacaagtctcaccatcggtcacgaaaaggccgcttcgagttcaatgatgtgtttgctattattaacgcgcttcccgctattgctcttatcaattatggattctccaatgaaggcctccttcctggagcgtgctttggtgtcggtcttggaacaacagtctgtggtatggcttacatttttcttcacaatggcctatcacaccgaaggttcccagtatggcttattgcgaacgtcccttatttccacaagctggctgcagctcaccaaatacaccactcaggaaaatttcagggtgtaccatttggcctgttccttggacccaaggaattggaagaagtaagaggaggcactgaagagttggagagggtaatcagtcgtacaactaaacgaacgcaaccatctacctgaatcaatttttttacatatataaggttttagtttatcggtgttataaaatcacacatccgtatcgttttagtaagtcaaagttaagatacttccttcttagaatattttttgatgtataggtcgcggatatactgttacactattcgttgtggaattccattataaaaaaataaaaaaaaaaaaaaaaaaa

(19) United States
(12) Patent Application Publication
(10) Pub. No.: US 2010/0088781 A1
(43) Pub. Date: Apr. 8, 2010

(54) ALTERING CAROTENOID PROFILES IN PLANTS

(75) Inventors: Abdelali Hannoufa, Saskatoon (CA); Derek J. Lydiate, Saskatoon (CA); Bianyun Yu, Saskatoon (CA); Ulrike A. Schäfer, Calgary (CA)

Correspondence Address: MORRISON & FOERSTER LLP, 425 MARKET STREET, SAN FRANCISCO, CA, 94105-2482, US

(73) Assignee: HER MAJESTY THE QUEEN IN RIGHT OF CANADA, AS REPRE SENTED BY THE MINISTER OF AGRICULTURE AND AGRIFOO, Saskatoon (CA)

(21) Appl. No.: 12/528,079

(22) PCT Filed: Feb. 21, 2008
(86) PCT No.: PCT/CA2008/000344

§ 371 Date: Dec. 8, 2009

Related U.S. Application Data
(60) Provisional application No. 60/966,544, filed on Feb. 21, 2007.

(51) Int. Cl. A01H 1/00; C07H 21/04; C12N 15/82; A01H 5/00; A01H 5/10

(52) U.S. Cl. 800/278; 536/24.5; 435/468; 800/298

(57) ABSTRACT

A method for altering the level of one or more carotenoids in a plant or a tissue within the plant is provided. Method comprises introducing a nucleic acid sequence comprising a regulatory region operatively associated with a silencing nucleotide sequence that reduces or eliminates the expression of a lycopene epsilon cyclase into the plant, and expressing the silencing nucleotide sequence. Expression of the sequence reduces the level of the lycopene epsilon cyclase in the plant or within a tissue of the plant, and results in altering one or more carotenoids.

### FIELD OF INVENTION

The present invention relates to methods of altering carotenoids within plants, and plants with increased carotenoid levels.

### BACKGROUND OF THE INVENTION

Carotenoids comprise a large group of secondary metabolites that are natural pigments present in most higher plants. They are essential components of photosynthetic membranes and provide photoprotection against light damage, by channeling excess energy away from chlorophyll. Carotenoids act as membrane stabilizers and are also possible precursors in abscisic acid biosynthesis. Carotenoids are synthesized and accumulated in the plastids of higher plants. Chloroplasts store carotenoids in thylakoid membranes associated with light harvesting, while chromoplasts may store high levels of carotenoids in membranes, oil bodies, or other crystalline structures within the stroma (Howitt and Pogson, 2006).

Carotenoids are derived from the isoprenoid pathway, in which the condensation of two geranylgeranyl diphosphate (GGDP) to form phytoene is the first committed step in carotenoid biosynthesis. Phytoene then undergoes four sequential desaturation reactions to form lycopene. In higher plants the cyclization of lycopene, involving lycopene β-cyclase (lycopene-beta cyclase) and lycopene ε-cyclase (lycopene epsilon-cyclase), is the branch point in carotenoid biosynthesis (see FIG. 1). On one branch a single enzyme, lycopene β-cyclase (β-CYC; lycopene beta cyclase; beta-CYC), introduces a β-(beta-) ring at both ends of lycopene to form β-carotene (beta-carotene) in a two step reaction. The first dedicated reaction in the other branch of the pathway, leading to lutein, requires both β-CYC (beta-CYC) and lycopene 6-cyclase (ε-CYC) to introduce one β-(beta-) and one ε-(epsilon-) ring into lycopene to form α-carotene (alpha-carotene; Cunningham and Gantt, 1998). A set of reactions in plants, the xanthophyll cycle, rapidly optimizes the concentration of zeaxanthin and violaxanthin in the cell through the action of zeaxanthin epoxidase and violaxanthin de-epoxidase via antheraxanthin (Demmig-Adams and Adams, 2002).

Carotenoids are widely used in the food and cosmetics industries for example as colourants (Fraser and Bramley, 2004; Taylor and Ramsay, 2005; Botella-Pavia and Rodriguez-Concepción, 2006), and their importance to human health has been well documented (Bartley and Scolnik, 1995; Mayne; 1996; Demmig-Adams and Adams, 2002; Krinsky and Johnson, 2005). For example, β-Carotene is the precursor of vitamin A (Lakshman and Okoh, 1993), and lutein and zeaxanthin provide protection against macular degeneration (Landrum and Bone, 2004). Vitamin A (retinol) deficiency in humans results in symptoms ranging from night blindness to total and irreversible blindness (Ye et al. 2000). The dietary consumption of foods rich in provitamin A (β-carotene) avoids deficiency. Lutein and zeaxanthin also help protect the eye by absorbing potentially harmful blue light radiation (Krinsky and Johnson, 2005).

In many crops used in human and animal diets, carotenoid levels are not adequate, and fortification of plants with these essential nutrients is needed. Botella-Pavía and Rodríguez-Concepción (2006) disclose metabolic engineering approaches to increase carotenoid concentrations in plants. Enhanced levels of both β-carotene and lutein were reported following tuber-specific expression of a bacterial phytoene synthase (PSY) gene in potato (Ducreux et al. 2005). Overexpression of an endogenous phytoene synthase in the seeds of Arabidopsis thaliana resulted in 43-fold average increase in the level of β-carotene (Lindgren et al., 2003). Rosati et al. (2000) teach that expression of A. thaliana lycopene β-CYC in tomato resulted in an increase in β-carotene content in tomato fruits. Expression of the Daffodil phytoene synthase (psy) and a bacterial phytoene desaturase (crtI) in rice resulted in the production of β-carotene, lutein and zeaxanthin (Ye et al. 2000).

Canola (Brassica napus) seed is a valuable source of oil for the food industry. In this process seed meal is produced and methods of increasing the value of this meal are desired. One approach of increasing value of the seed meal is to increase carotenoid levels within canola seeds. Shewmaker et al. (1999) teach the overexpression of a bacterial phytoene synthase (PSY, also known as crtB) in a seed-specific manner in Brassica napus. This resulted in a 50-fold increase in carotenoid, concentrations, especially beta-carotene, with little to no change in lutein concentration. However, the fatty acid profile of the seed oil was altered with increases in several fatty acids including 18:0, 20:0, and a decrease in 18:3 fatty acids, and this may reduce the utility of the seed oil. Ravanello et al (2003) disclose the over-expression of crtB along with enzymes involved in the carotenoid pathway, including crtE (geranylgeranyl diphosphate synthase), crtI (phytoene desaturase), or crtY (lycopene cyclase).

Alternate methods to increase carotenoid levels in seed, preferably without altering the fatty acid profile are desired

### SUMMARY OF THE INVENTION

The present invention relates to methods of altering carotenoids within plants, and plants with increased carotenoid levels.

The present invention provides a method (method A) to increase the levels of carotenoids in seed comprising,

i) providing a plant comprising a nucleotide sequence that inhibits the expression of endogenous ε-CYC (lycopene epsilon cyclase), and

ii) growing the plant under conditions that permit the expression of the nucleotide sequence thereby increasing the levels of carotenoids in the seed.

The seed may be obtained following the step of growing (step ii), and the carotenoids purified, oil extracted, or both the carotenoids and oil may be obtained.

The endogenous ε-CYC gene may be inhibited by RNAi, ribozyme, antisense RNA, or a transcription factor. Furthermore the portion of the ε-CYC gene that is targeted is specific to the ε-CYC gene, for example using 5′, 3′; or both 5′ and 3′ specific regions of ε-CYC.

The present invention also provides a method (method B) for altering the level of one or more carotenoids in a plant or a tissue within the plant comprising,

i) introducing a nucleic acid sequence into the plant, the nucleic acid sequence comprising a regulatory region operatively associated with a silencing nucleotide sequence, wherein expression of the silencing nucleotide sequence reduces or eliminates the expression of a lycopene epsilon cyclase (ε-CYC), and

ii) expressing the silencing nucleotide sequence within the plant or a tissue within the plant to reduce the level of the lycopene epsilon cyclase (ε-CYC) in the plant or within a tissue of the plant, thereby altering the level of the one or more carotenoid in the plant or plant tissue, the reduced level of lycopene epsilon cyclase determined by comparing the level of expression of the lycopene epsilon cyclase in the plant, or a tissue of the plant, with a level of lycopene epsilon cyclase in a second plant, or the tissue from the second plant, that does not express the silencing nucleic acid sequence.

The silencing nucleotide sequence as described in method B may be selected from the group consisting of an antisense RNA encoding nucleotide sequence, a ribozyme encoding sequence, and an RNAi encoding nucleotide sequence. Furthermore, the regulatory region may be selected from the group consisting of a constitutive regulatory region, an inducible regulatory region, a developmentally regulated regulatory region, and a tissue specific regulatory region. For example, the regulatory region is a tissue specific regulatory region.

The present invention also pertains to the method describe above (method B), wherein the level of the one or more than one carotenoid is reduced by about 25 to about 100%, where compared to the level of the same one or more than one carotenoid obtained from second plant.

The present invention includes a method as described above (method B), wherein, the silencing nucleotide sequence reduces the level of expression of lycopene epsilon cyclase (ε-CYC), while the level of expression of lycopene beta cyclase (β-CYC) remains similar to that of a second plant, or the tissue from the second plant, that does not express the silencing nucleotide sequence, and the reduced level of lycopene epsilon cyclase determined by comparing the level of expression of the lycopene epsilon cyclase in the plant, or a tissue of the plant, with a level of lycopene epsilon cyclase in the second plant, or the tissue from the second plant, that does not express the silencing nucleic acid sequence. For example, the silencing nucleotide sequence may be selected from the group of SEQ ID NO:2, SEQ ID NO:3, nucleotides 76-427 of SEQ ID NO:1, 1472-1881 of SEQ ID NO:1, 28-384 of SEQ ID NO:4, and 1411-1835 of SEQ ID NO:4, or a nucleotide sequence that hybridizes to SEQ ID NO:2, SEQ ID NO:3, nucleotides 76-427 of SEQ ID NO:1, and 1472-1881 of SEQ ID NO:1, 28-384 of SEQ ID NO:4, and 1411-1835 of SEQ ID NO:4, or that hybridizes to a complement of SEQ ID NO:2, SEQ ID NO:3, nucleotides 76-427 of SEQ ID NO:1, and 1472-1881 of SEQ ID NO:1, 28-384 of SEQ ID NO:4, and 1411-1835 of SEQ ID NO:4, under stringent hybridization conditions, the stringent hybridization conditions comprising hybridization in Church buffer at 61° C. for 22 hr, washing the filter twice in 2×SSC, 0.1% SDS for 10 min at 61° C., and washing twice in 0.2×SSC, 0.1% SDS for 10 min at 61° C., wherein the silencing nucleotide sequence exhibits reduces expression of a lycopene epsilon cyclase (ε-CYC) gene or sequence from about 10 to about 100%.

The present invention also provides a nucleic acid sequence comprising, a regulatory region operatively associated with a silencing nucleotide sequence that reduces or eliminates the expression of a lycopene epsilon cyclase (ε-CYC), and does not alter the level of expression of lycopene beta cyclase (β-CYC). The silencing nucleotide sequence may be selected from the group consisting of an antisense RNA encoding nucleotide sequence, a ribozyme encoding sequence, and an RNAi encoding nucleotide sequence. For example, the silencing nucleotide sequence may be selected from the group of nucleotides 76-427 of SEQ ID NO:1, 1472-1881 of SEQ ID NO:1, 28-384 of SEQ ID NO:4, and 1411-1835 of SEQ ID NO:4, or a nucleotide sequence that hybridizes to nucleotides 76-427 of SEQ ID NO:1, 1472-1881 of SEQ ID NO:1, 28-384 of SEQ ID NO:4, and 1411-1835 of SEQ ID NO:4, or that hybridizes to a complement of nucleotides 76-427 of SEQ ID NO:1, 1472-1881 of SEQ ID NO:1, 28-384 of SEQ ID NO:4, and 1411-1835 of SEQ ID NO:4, under stringent hybridization conditions, the stringent hybridization conditions comprising hybridization in Church buffer at 61° C. for 22 hr, washing the filter twice in 2×SSC, 0.1% SDS for 10 min at 61° C., and washing twice in 0.2×SSC, 0.1% SDS for 10 min at 61° C., wherein the silencing nucleotide sequence exhibits reduces expression of a lycopene epsilon cyclase (ε-CYC) gene or sequence from about 10 to about 100%. The regulatory region may be selected from the group consisting of a constitutive regulatory region, an inducible regulatory region, a developmentally regulated regulatory region, and a tissue specific regulatory region. For example, the regulatory region is a tissue specific regulatory region.

The present invention also provides a construct comprising the nucleic acid sequence as just defined above, a plant comprising the nucleic acid sequence as just defined above, and a seed comprising the nucleic acid sequence, as just defined above.

Mutant plant lines with knockouts in genes affecting ε-CYC expression were characterized and found to exhibit increased levels of carotenoids, including beta carotene and lutein, while at the same time the fatty acid profile remained essentially unaltered when compared to wild type fatty acid profile. The approach is exemplified using B. napus, however, other plants may also be modified using the methods as described herein, for example, but not limited to canola, Brassica spp., B. carinata, B. nigra, B. oleracea, B. chinensis, B. cretica, B. incana, B. insularis, B. japonica, B. atlantica, B. bourgeaui, B. narinosa, B. juncea, B. rapa, Arabidopsis thaliana, soybean, corn, barley, wheat, buckwheat, rice, tobacco, alfalfa, potato, ginseng, pea, oat, cotton, sunflower, and other oil seed plants.

To enhance the level of carotenoids in the seed, the expression of ε-CYC was downregulated using RNAi. Inactivation of ε-CYC led to an increase in the levels of carotenoids including β-carotene, lutein and violaxanthin in B. napus seeds. Transgenic seeds exhibited slight reductions in lipid content and minor alterations in fatty acid profiles relative to the wild type control.

The present invention also provides a method (method C) for altering the carotenoid profile in a plant or a tissue within the plant comprising,

i) providing the plant comprising:

a) a first nucleic acid sequence comprising a regulatory region operatively associated with a silencing nucleotide sequence, wherein expression of the silencing nucleotide sequence reduces or eliminates the expression of a lycopene epsilon cyclase, and
b) one or more than one second nucleic acid sequence, wherein each of the one or more than one second nucleic acid sequence comprise a regulatory region operatively associated with a sequence that encodes one or more than one enzyme involved in carotenoid synthesis, and

ii) expressing the silencing nucleotide sequence and the one or more than one second nucleic acid sequence within the plant or a tissue within the plant, wherein expression of the silencing nucleotide sequence reduce the level of the lycopene epsilon cyclase in the plant or within a tissue of the plant, the reduced level of lycopene epsilon cyclase may be determined by comparing the level of expression of the lycopene epsilon cyclase in the plant, or a tissue of the plant, with a level of the lycopene epsilon cyclase in a second plant, or the tissue from the second plant, that does not express the silencing nucleic acid sequence, and expression of the one or more than one second nucleic acid sequence results in increased expression of a the one or more than one enzyme involved in carotenoid synthesis.

Examples of one or more than one additional nucleotide sequence that may be coexpressed in a plant as outlined above include, but are not limited to beta carotene hydroxylase, beta carotene 3-hydroxylase, beta-carotene ketolase, phytoene synthase, phytoene desaturase, zeaxanthin epoxidase.

The present invention also provides a method (method D) for altering the level of one or more carotenoid in a plant or a tissue within the plant comprising,

i) providing a plant expressing a nucleic acid sequence, the nucleic acid sequence comprising a regulatory region operatively associated with beta carotene hydroxylase, beta-carotene, ketolase, or beta carotene hydroxylase and beta-carotene, ketolase, and

ii) growing the plant under conditions that express the nucleic sequence within the plant or a tissue within the plant thereby altering the level of the one or more carotenoid in the plant or plant tissue.

The tissue may be seed tissue, and the regulatory region may be a seed specific promoter, or a constitutive promoter.

The present invention includes the method as described above (method D), wherein the beta carotene hydroxylase is crtH1, and the beta-carotene, ketolase is adketo2.

Enhanced levels of carotenoids, including β-carotene, lutein and violaxanthin, zeaxanthin and beta-cryptoxanthin were obtained in the seed of B. napus plants, following the selective downregulation of the expression of ε-CYC. As these transgenic seeds exhibited only slight reductions in lipid content and minor alterations in fatty acid profiles relative to the wild type control (Table 4), these seeds may be used to obtain canola quality oil, while at the same time be used to obtain increased levels of carotenoids.

There is increasing interest in using plant-based diets as a replacement for expensive and poorly sustainable fish meal in aquaculture feeds. Canola (Brassica napus) seed offers a sustainable alternative to conventional fish meal due to the good amino acid balance of its proteins, low cost compared to conventional fish meal, high availability and local production. However, in addition to its high content of antinutritional factors, B. napus seed also lacks the carotenoid pigment, astaxanthin. This is an expensive fish feed supplement, and therefore producing a B. napus seed that contains astaxanthin is beneficial to both aquaculturalists and producers.

This summary of the invention does not necessarily describe all features of the invention.

### BRIEF DESCRIPTION OF THE DRAWINGS

FIG. 1 shows a schematic chart of carotenoid biosynthesis in plants

FIG. 2A shows a sequence alignment between ε-CYC (epsilonCYC; NM_{—}125085; SEQ ID NO:4), and β-CYC (beta CYC; NM_{—}111858; SEQ ID NO:28) from Arabidopsis thaliana. Identical nucleotide sequences are shown as white letters on gray background. 5′- and 3′-ends of Brassica napus ε-CYC were aligned to 28-384 by and 1411-1835 by of Arabidopsis epsilon CYC, NM_{—}125085 respectively. FIG. 2B shows the Brassica napus lycopene epsilon cyclase cDNA 5′-end (SEQ ID NO:2). FIG. 2C shows the Brassica napus lycopene epsilon cyclase cDNA 3′-end (SEQ ID NO:3). FIG. 2D shows the Brassica napus lycopene epsilon cyclase sequence (SEQ ID NO:1). FIG. 2E shows a sequence alignment of the 5′ region between lycopene epsilon cyclase (SEQ ID NO:35; or nucleotides 1-400 of SEQ ID NO:1) and lycopene beta cyclase from B. napus. The 5′ region exhibits a 26.4% sequence identity. FIG. 2F shows a sequence alignment of the 3′ region between lycopene epsilon cyclase (SEQ ID NO:36; or nucleotides 1471-1984 of SEQ ID NO:1) and lycopene beta cyclase from B. napus. The 3′ region exhibits a 29.9% sequence identity. FIG. 2G shows a sequence alignment of the mid region between lycopene epsilon cyclase (SEQ ID NO:34; or nucleotides 429-1470 of SEQ ID NO:1) and lycopene beta cyclase from B. napus. The mid region exhibits a 51.7% sequence identity.

FIG. 3 shows a diagrammatic representation of RNAi constructs 710-422 comprising a 352 base pair fragment from the 5′ region of lycopene epsilon cyclase, and 710-423 comprising a 410 base pair region from the 3′ end of lycopene epsilon cyclase (see examples for details). Sequences were PCR amplified from the 5′ and 3′ ends of a B. napus lycopene epsilon-cyclase EST and used to generate the RNAi constructs.

FIG. 4 shows expression profiles of carotenoid biosynthesis genes in different organs, and in developing seeds of B. napus. FIG. 4a shows RT-PCR fragment amplified from templates of cDNA (1) and genomic DNA (2). FIG. 4b shows gene expression in different organs of B. napus relative to a co-amplified actin internal control. FIG. 4c shows gene expression in developing B. napus seeds relative to a co-amplified actin internal control. PSY, phytoene synthase; PDS, phytoene desaturase; beta-CYC, lycopene, beta-cyclase; epsilon-CYC, lycopene epsilon-cyclase; DPA, days post-anthesis.

FIG. 5 shows gene expression in developing seeds of select epsilon-CYC RNAi lines (BY351, BY371); DH12075, untransformed control. PSY, phytoene synthase; PDS, phytoene desaturase; beta-CYC, lycopene beta-cyclase; epsilon-CYC, lycopene epsilon-cyclase.

FIG. 6 shows carotenoid extracts from dry mature seeds of epsilon-CYC-RNAi lines BY54, BY223, BY365 and the untransformed control DH12075 line

FIG. 7 shows Southern blot analysis of epsilon-CYC gene family in B. napus. Approximately 10 μg of genomic DNA was digested with BamHI, EcoRI, EcoRV, SalI, SpeI and SstI restriction endonucleases. The blot was probed with a 352 by B. napus epsilon-CYC cDNA fragment. Size markers (bp) are indicated.

FIG. 8 shows a diagrammatic representation of additional constructs. FIG. 8a shows construct 710-433, comprising a 930 bp ORF fragment of CrtH1 (encoding beta carotene hydroxylase) obtained from Adonis aestivalis. FIG. 8b shows construct 710-438, comprising a 940 by ORF fragment of Adketo2 (encoding beta carotene 3-hydroxylase) prepared from Adonis aestivalis. FIG. 8c shows construct 710-440A comprising the 940 by ORF of Adketo2 and the 930 by ORF from CrtH1.

FIG. 9 shows a diagrammatic representation of vector 70-103 harbouring a BAR gene for glyphosinate selection in plants.

FIG. 10A shows the nucleotide sequences of crtH1 obtained from Adonis aestivalis (SEQ ID NO:37). FIG. 10 B shows a alignment of amino acid sequences of beta-carotene hydroxylases of various organisms, and positions of the degenerate primers used to amplify the conserved 363 by fragment. Identical and highly conserved amino acids in the six sequences are shown as white letters on black and gray backgrounds, and amino acids with similarity are indicated as black letters on a gray background. Amino acids with no similarity are shown as black letters on a white background. GenBank accession numbers of these sequences are as follows: Lycopersicon esculentum LeCrtR-b1 (Y14809) and LeCrtR-b2 (Y14810); Alcaligenes sp. AsCrtZ (D58422); Arabidopsis thaliana AtHX1 (AF370220); Citrus unshiu CHX1 (AF296158); Haematococcus pluvialis HpHX (AF162276). FIG. 10C shows the nucleotide sequence of adketo2 obtained from Adonis aestivalis (SEQ ID NO:38).

FIG. 10D shows Northern analysis of CrtH1 in immature siliques of transgenic Arabidopsis thaliana. Upper panel A shows wild type (wt) and wild type expressing CrtH1 (BY275 to BY284), lower panel B shows b1b2 mutant, and b1b2 mutant expressing CrtH1 (BY317 to BY347). FIG. 10E shows HPLC profiles of carotenoids extracted from seeds of Arabidopsis thaliana. Panel a: Wild type expressing CrtH1 (BY287line), panel b: wild type, panel c: b1b2 mutant expressing CrtH1 (BY317line) and panel d: b1b2 mutant. Peaks numbered 1, 2, 3, 4 and 5 correspond to violaxanthin, lutein, zeaxanthin, beta-cryptoxanthin and beta-carotene, respectively. FIG. 10F shows HPLC profiles of carotenoid extracts from seeds of B. napus DH12075 parental line (top panel), and line DE1339 expressing p710-440 construct harboring both crtH1 and adKeto2 (bottom panel). Astaxanthin peak is circled.

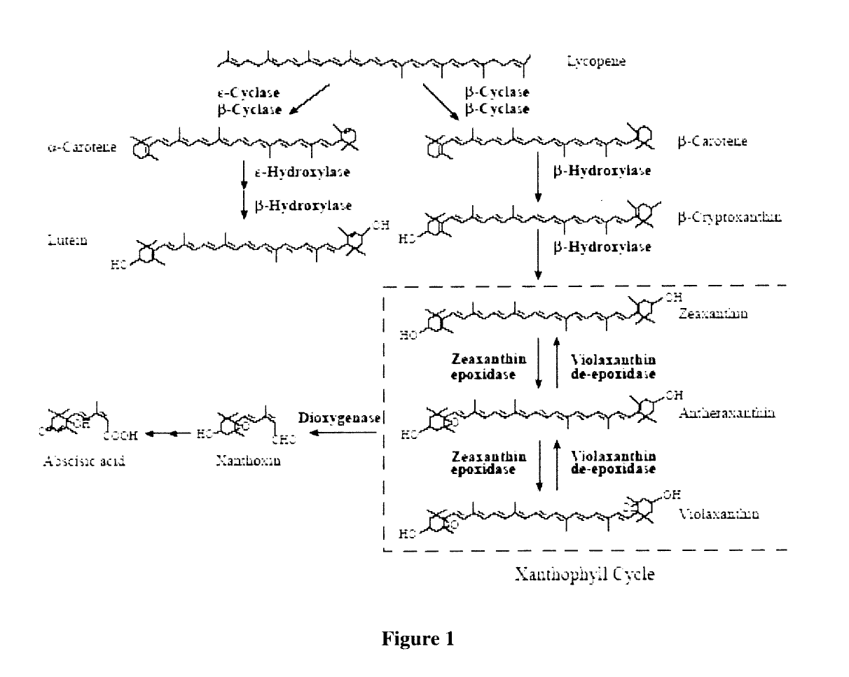

### DETAILED DESCRIPTION

The present invention relates to methods of altering carotenoids within plants, and plants with increased carotenoid levels.

The present invention provides a method to alter the levels of carotenoids in seeds, for example, to increase the levels of carotenoids in seeds The method involves providing a plant comprising a nucleotide sequence that inhibits the expression of endogenous ε-CYC (lycopene epsilon cyclase), for example SEQ ID NO:1 (B. napus epsilon CYC), a sequence that exhibits from about 80 to about 100% sequence identity with SEQ ID NO:1 provided that the nucleotide sequence retains the property of silencing expression of a lycopene epsilon cyclase (ε-CYC) gene or sequence, or a sequence that hybridizes to SEQ ID NO:1 under stringent conditions as defined below, again provided that the nucleotide sequence retains the property of silencing expression of a lycopene epsilon cyclase (ε-CYC) gene or sequence, and growing the plant under conditions that permit the expression of the nucleotide sequence. By specifically inhibiting ε-CYC, the levels of carotenoids in general in the seed are increased, including β-carotene and lutein. Using the methods described herein, the increase is not limited to β-carotene. Seed may be obtained from such plants, the carotenoids purified, and the oil extracted, or both the carotenoids and oil may be obtained from the seed.

The present invention provides a method for altering the level of one or more than one carotenoid in a plant or a tissue within the plant comprising,

i) providing the plant comprising a nucleic acid sequence comprising a regulatory region operatively associated with a silencing nucleotide sequence, wherein expression of the silencing nucleotide sequence reduces or eliminates the expression of a lycopene epsilon cyclase, and

ii) expressing the silencing nucleotide sequence within the plant or a tissue within the plant, to reduce the level of the lycopene epsilon cyclase in the plant or within a tissue of the plant, the reduced level of lycopene epsilon cyclase may be determined by comparing the level of expression of the lycopene epsilon cyclase in the plant, or a tissue of the plant, with a level of the lycopene epsilon cyclase in a second plant, or the tissue from the second plant, that does not express the silencing nucleic acid sequence.

The endogenous ε-CYC (lycopene epsilon cyclase) gene may be inhibited by RNAi, ribozyme, antisense RNA or a transcription factor, for example, a native transcription factor, or a synthetic transcription factor. Furthermore the ε-CYC gene that is targeted for inhibition or silencing within the plant may be inhibited or silenced using a portion of ε-CYC gene, for example by using a 5′, a 3′; or both 5′ and 3′ specific regions of ε-CYC. Examples of 5′ or 3′ regions of lycopene epsilon cyclase gene that may be used for silencing include the nucleotide sequence defined in SEQ ID NO:2 (5′ region of lycopene epsilon cyclase), and the nucleotide sequence defined in SEQ ID NO:3 (3′ region of lycopene epsilon cyclase), a nucleotide sequence that exhibits from about 80 to about 100% sequence identity to the nucleotide sequence defined in SEQ ID NO:2 (5′ region of lycopene epsilon cyclase), a nucleotide sequence that exhibits from about 80 to about 100% sequence identify to the nucleotide sequence defined in SEQ ID NO:3 (3′ region of lycopene epsilon cyclase), a nucleotide sequence that hybridizes to the nucleotide sequence defined in SEQ ID NO:2 (5′ region of lycopene epsilon cyclase) or its complement, under stringent hybridization conditions as defined below, or a nucleotide sequence that hybridizes to the nucleotide sequence defined in SEQ ID NO:3 (3′ region of lycopene epsilon cyclase) or its complement, under stringent hybridization conditions, as defined below.

The lycopene epsilon cyclase may be from any source provided that it exhibits the sequence identity as defined above, or hybridizes in a manner as described above. For example the lycopene epsilon cyclase may be obtained from a plant, for example but not limited to B. napus, or Arabidopsis, a tree, a bacteria, an algae, or a fungus.

The ε-CYC gene that is targeted for inhibition or silencing within the plant may be inhibited or silenced using a portion of ε-CYC gene for example from B. napus comprising nucleotides 76-427 of SEQ ID NO:1, 1472-1881 of SEQ ID NO:1, or both 76-427 of SEQ ID NO:1 and 1472-1881 of SEQ ID NO:1, or from A. thaliana, comprising nucleotides 28-384 of SEQ ID NO:4, 1411-1835 of SEQ ID NO:4, or both 28-384 of SEQ ID NO:4 and 1411-1835 of SEQ ID NO:4, or a nucleotide sequence that exhibits from about 80 to about 100% sequence identity to the nucleotide sequence of 76-427 of SEQ ID NO:1, 1472-1881 of SEQ ID NO:1, 28-384 of SEQ ID NO:4, 1411-1835 of SEQ ID NO:4, or a nucleotide sequence that hybridizes to the nucleotide sequence defined by nucleotides 76-427 of SEQ ID NO:1, or its complement, 1472-1881 of SEQ ID NO:1, or its complement, 76-427 of SEQ ID NO:1, or its complement, and 1472-1881 of SEQ ID NO:1, or its complement, or nucleotides 28-384 of SEQ ID NO:4, or its complement, 1411-1835 of SEQ ID NO:4, or its complement, 28-384 of SEQ ID NO:4, or its complement, 1411-1835 of SEQ ID NO:4, or its complement, under stringent hybridization conditions as defined below.

The present invention therefore provides a method for increasing the concentration of carotenoids in a plant or a tissue within the plant comprising, providing a plant in which the activity of lycopene epsilon-cyclase or the expression of nucleotide sequence encoding lycopene epsilon cyclase is selectively reduced when compared to the activity of lycopene epsilon cyclase or the expression nucleotide sequence encoding lycopene epsilon cyclase, as measured within a second plant comprising wild-type levels of lycopene epsilon-cyclase, or wild type expression levels of the nucleotide sequence encoding lycopene epsilon cyclase. The lycopene epsilon cyclase activity, or the expression of the nucleotide sequence encoding lycopene epsilon cyclase may also be reduced within a plant in a tissue-specific manner, for example, the levels may be reduced within mature seed tissue.

The level of the lycopene beta cyclase activity, or the expression of the nucleotide sequence encoding lycopene epsilon cyclase, within a plant may be reduced by inhibiting the expression of the cyclase for example by inhibiting transcription of the gene encoding lycopene epsilon cyclase, reducing levels of the transcript, or inhibiting synthesis of the lycopene epsilon cyclase protein. The levels of lycopene epsilon cyclase may be inhibited from about 10% to about 100%, or any amount therebetween, where compared to the level of lycopene beta cyclase obtained from a second plant that expresses the nucleotide sequence at wild-type levels. For example, the protein may be reduced by from about 10% to about 80% or any amount therebetween, about 10% to about 50% or any amount therebetween, about 10% to about 40% or any amount therebetween, from about 10% to about 30%, or any amount therebetween, about 10% to about 20% or any amount therebetween, or about 10, 15, 20, 25, 30, 35, 40, 45, 50, 55, 60, 65, 70, 76, 80, 85, 90, 95 or 100%, or any amount therebetween. Furthermore, the level of the nucleotide encoding lycopene epsilon cyclase may be inhibited from about 10% to about 100%, or any amount therebetween, where compared to the level of the nucleotide encoding lycopene beta cyclase obtained from a second plant that expresses the nucleotide sequence at wild-type levels. For example, the expression of the nucleotide sequence may be reduced by from about 10% to about 80% or any amount therebetween, about 10% to about 50% or any amount therebetween, about 10% to about 40% or any amount therebetween, from about 10% to about 30%, or any amount therebetween, about 10% to about 20% or any amount therebetween, or about 10, 15, 20, 25, 30, 35, 40, 45, 50, 55, 60, 65, 70, 76, 80, 85, 90, 95 or 100%, or any amount therebetween.

The regulatory region may be a constitutive regulatory region, an inducible regulatory region, a developmentally regulated regulatory region, or a tissue specific regulatory region.

By “operatively linked” or “operatively associated” it is meant that the particular sequences interact either directly or indirectly to carry out an intended function, such as mediation or modulation of expression. The interaction of operatively linked sequences may, for example, be mediated by proteins that interact with the operatively linked sequences. A coding region of interest may also be introduced within a vector along with other sequences, that may be heterologous, to produce a chimeric construct.

By the term “expression” it is meant the production of a functional RNA, protein or both, from a nucleotide sequence, a gene or a transgene.

By “reduction of gene expression” or reduction of expression” it is meant the reduction in the level of mRNA, protein, or both mRNA and protein, encoded by a gene or nucleotide sequence of interest. Reduction of gene expression may arise as a result of the lack of production of full length RNA, for example mRNA, or through cleaving the mRNA, for example with a ribozyme (e.g. see Methods in Molecular Biology, vol 74 Ribozyme Protocols, P.C. Turner, ed, 1997, Humana Press), or RNAi (e.g. see Gene Silencing by RNA Interference, Technology and Application, M. Sohail ed, 2005, CRC Press; Fire A, et al, 1998, Horiguchi G, 2004; Wesley et al. 2001), or otherwise reducing the half-life of RNA, using antisense (e.g. see Antisense Technology, A Practical Approach, C. Lichtenstien and W. Nellen eds., 1997, Oxford University Press), ribozyme, RNAi techniques, or by using a natural or synthetic transcription factor that is targeted to the promoter and results in the down regulation of lycopene epsilon cyclase.

A “silencing nucleotide sequence” refers to a sequence that when transcribed results in the reduction of expression of a target gene, or it may reduce the expression of two or more than two target genes, for example, 1, 2, 3, 4, 5, 6, 7, 8, 9, 10 target genes, or any number of target genes therebetween. A silencing nucleotide sequence may involve the use of antisense RNA, a ribozyme, or RNAi, targeted to a single target gene, or the use of antisense RNA, ribozyme, or RNAi, comprising two or more than two sequences that are linked or fused together and targeted to two or more than two target genes. When transcribed the product of the silencing nucleotide sequence may target one, or it may target two or more than two, of the target genes. When two or more than two sequences are linked or fused together, these sequences may be referred to as gene fusions, or gene stacking. It is within the scope of the present invention that gene fusions may comprise 2, 3, 4, 5, 6, 7, 8, 9, 10 nucleotide sequences, or any number therebetween, that are fused or linked together. The fused or linked sequences may be immediately adjacent each other, or there may be linker fragment between the sequences. Reduction in the expression of a lycopene epsilon cyclase (ε-CYC), results in the reduced synthesis of a protein encoded by the lycopene epsilon cyclase.

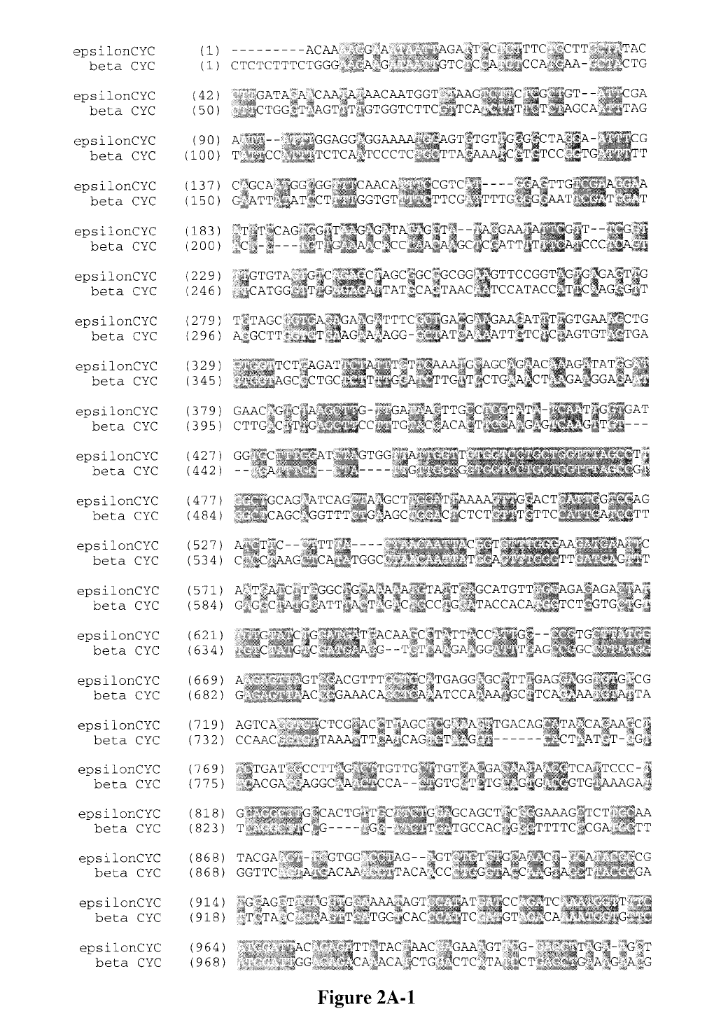

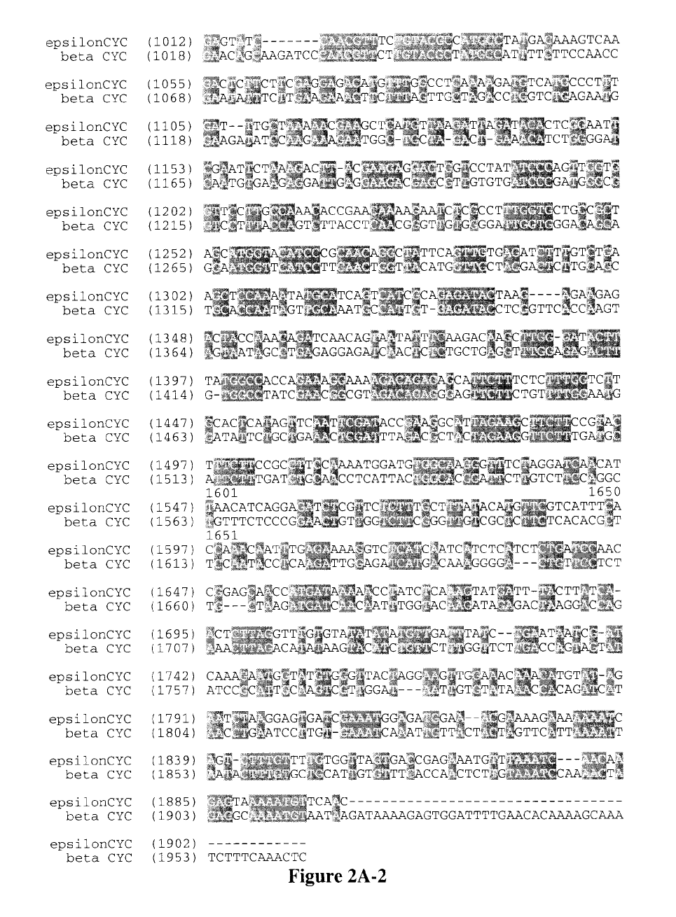

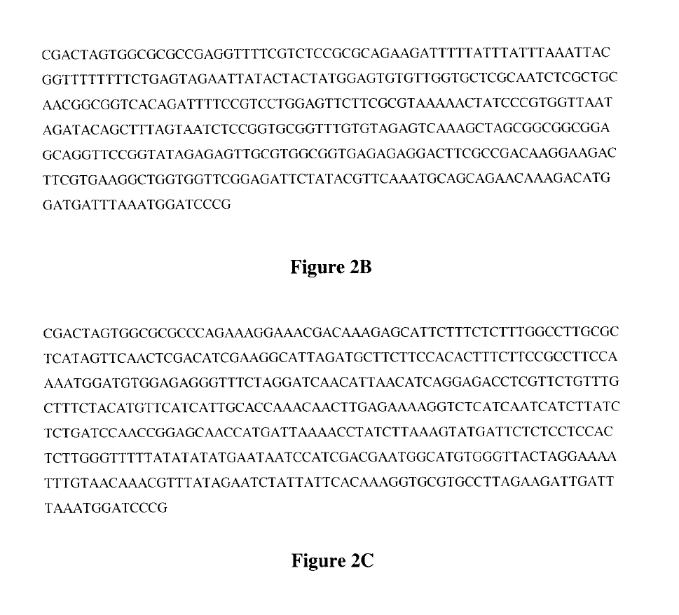

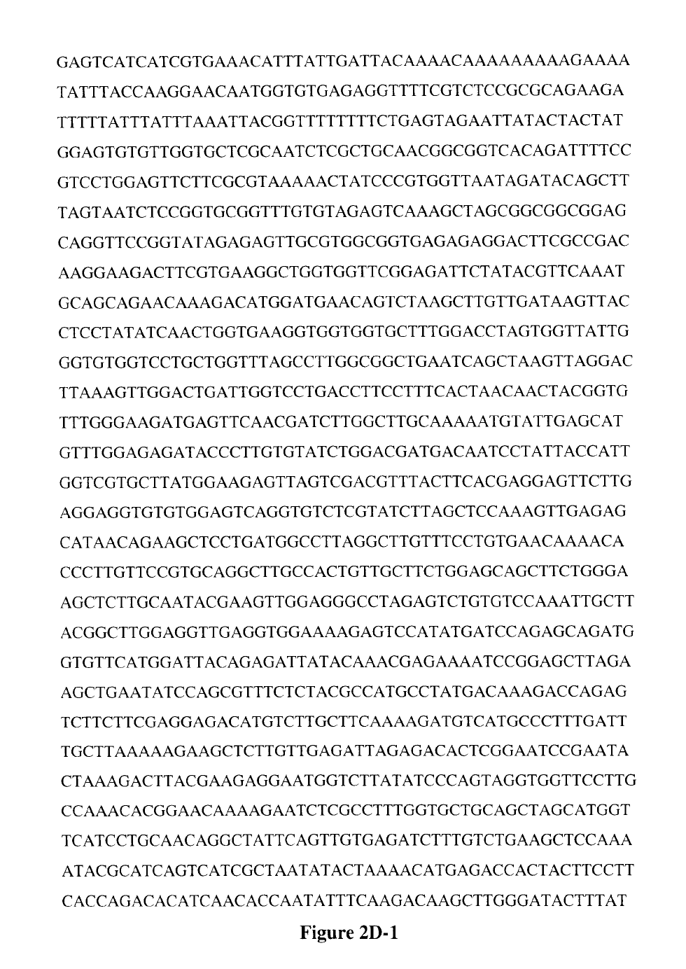

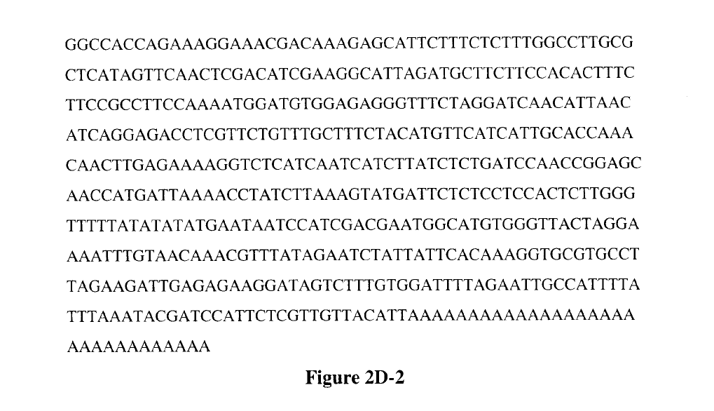

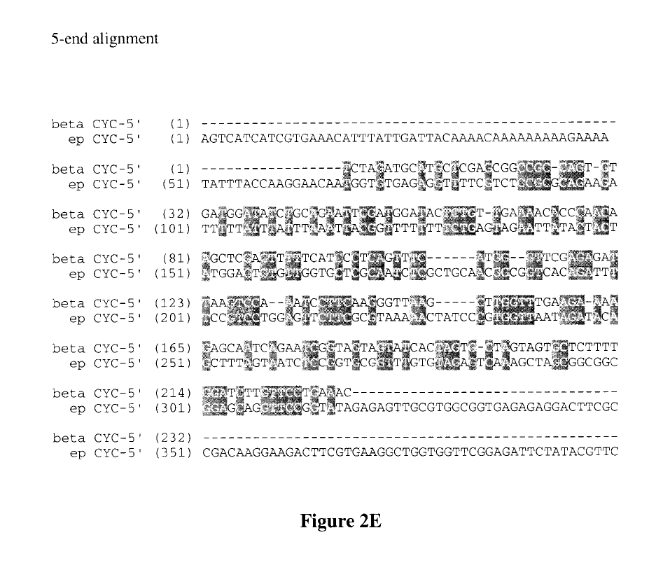

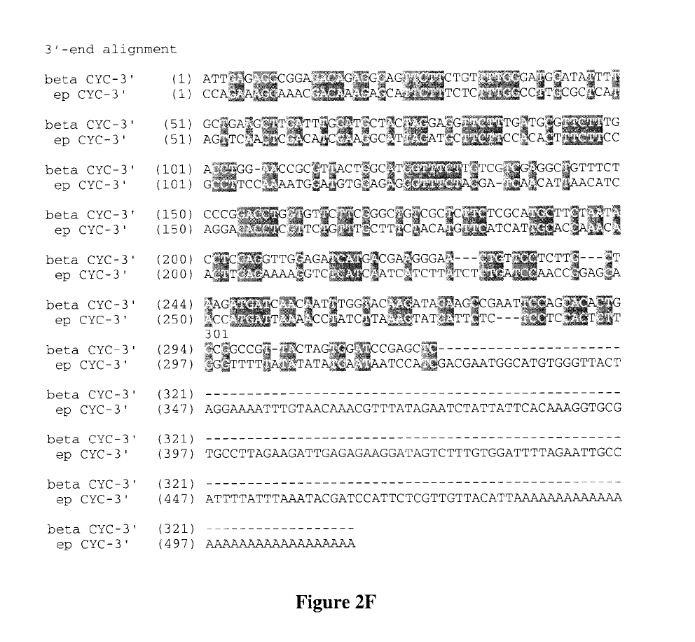

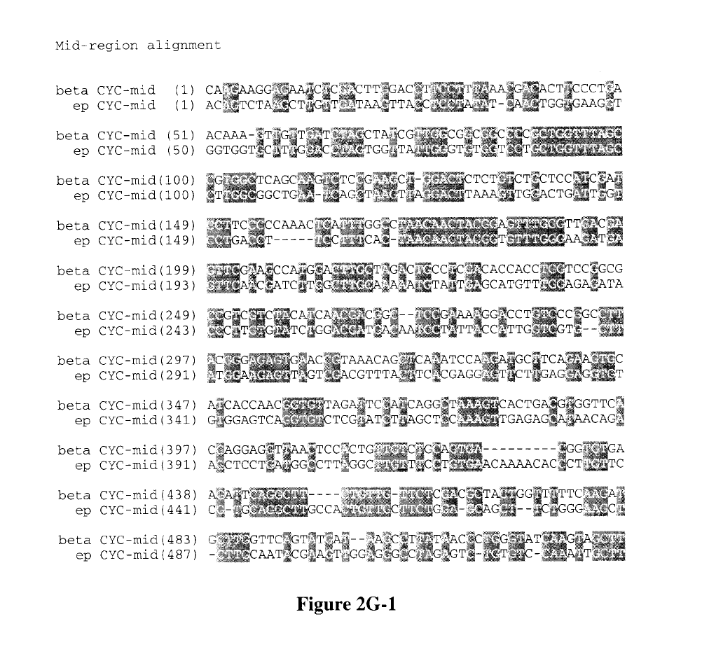

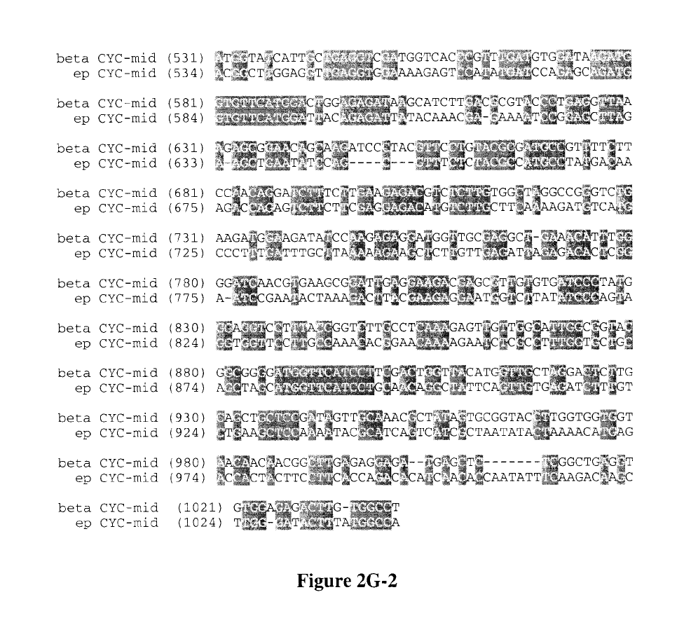

When the activity of ε-CYC is to be preferentially reduced, a nucleotide sequence that is specific for the 5′, 3′, or both 5′ and 3′ regions of the ε-CYC gene may be used. These regions of ε-CYC exhibit reduced sequence homology when compared to other cyclase genes, including for example β cyclase (beta-cyclase; see FIGS. 2A, 2E, 2F and 2G). The 5′ region of lycopene epsilon cyclase, as shown in FIG. 2E, exhibits a 26.4% sequence identity with the 5′ region of lycopene beta cyclase; the 3′ region of lycopene epsilon cyclase exhibits a 29.9% sequence identity with the 3′ region of lycopene beta cyclase (FIG. 2F); and the mid region of lycopene epsilon cyclase exhibits a 51.7% sequence identity with the mid region of lycopene beta cyclase. Prior art strategies to reduce ε-CYC in plants, for example as used in U.S. Pat. No. 6,653,530, involved the use of an antisense RNA construct that included a 903 nucleotide fragment of the gene sequence of the ε-CYC gene (a XhoI-BamHI fragment of B. napus ε-CYC sequence. The sequence comprising nucleotides 52-955 of SEQ ID NO:1 exhibits a high degree of similarity with other cyclase genes including β cyclase (see FIG. 2A). Without wishing to be bound by theory, the use of an antisense construct directed to the XhoI-BamHI of B. napus ε-CYC sequence may reduce the expression of not only ε-CYC (lycopene epsilon cyclase) but also other lycopene cylases including β cyclase (lycopene beta cyclase) genes.

The use of the silencing nucleic acids as described herein did not result in a reduction of lycopene epsilon cyclase expression (see FIG. 5, beta-CYC, v. epsilon-CYC).

In the present invention the activity of ε-CYC is selectively or preferentially inhibited. As a result of this preferential inhibition, it is has been observed that the carotenoid levels in seed tissue of both beta carotene and lutein are increased. This is very different from the finding disclosed in U.S. Pat. No. 6,653,530 which demonstrates a selective increase in beta carotene levels with a negligible change in lutein.

By “preferential inhibition” or “selective inhibition” it is meant that the expression of the target nucleotide sequence is inhibited by about 10 to about 100% when compared to the expression of a reference sequence. For example, the expression of the desired sequence may be inhibited by about 20 to about 80%, or any amount therebetween, or 20-50%, or any amount therebetween, when compared to the expression of the same sequence in a plant of the same variety (or genetic background) that does not express a silencing sequence, for example a wild-type plant, or when compared to the expression of a reference sequence in the same plant. For example, the expression of the desired sequence may be inhibited by about 10, 20, 30, 40, 50, 60, 70, 80, 90 100% or any amount therebetween, when compared to the expression of the same sequence, in a plant of the same variety (or genetic background) that does not express a silencing sequence, for example a wild-type plant, or when compared to the expression of a reference sequence in the same plant. A non-limiting example of a desired sequence is ε-CYC, and a reference sequence is β cyclase. In this case, preferential (or selective) inhibition of ε-CYC is achieved when the expression of ε-CYC is inhibited by about 10, 20, 30, 40, 50, 60, 70, 80, 90 100% or any amount therebetween, when compared to the expression of β cyclase, in the same plant, or when the expression of ε-CYC is inhibited by about 10, 20, 30, 40, 50, 60, 70, 80, 90 100% or any amount therebetween, when compared to the expression of lycopene epsilon cyclase, in a wild-type plant of the same genetic background.

Non-limiting examples of one or more than one silencing nucleotide sequence includes SEQ ID NO:2 (5′ region of ε-CYC), SEQ ID NO:3 (3′ portion of ε-CYC), or a combination of the 5′ and 3′ regions of ε-CYC (SEQ ID NO:2 and SEQ ID NO:3). Additional examples of a silencing nucleotide sequence include a nucleotide sequence that is from about 80 to about 100% similar, or any amount therebetween, or 80, 85, 90, 95 or 100% similar, as determined by sequence alignment of the nucleotide sequences as defined below, to SEQ ID NO:2 (5′ region of ε-CYC), SEQ ID NO:3 (3′ portion of ε-CYC), or a combination of the 5′ and 3′ regions of ε-CYC (SEQ ID NO:2 and SEQ ID NO:3). Alternatively, an example of a silencing nucleotide sequence includes a nucleotide sequence or that hybridizes under stringent hybridization conditions, as defined below, to SEQ ID NO:2 (5′ region of ε-CYC), SEQ ID NO:3 (3′ portion of ε-CYC), or a combination of the 5′ and 3′ regions of ε-CYC (SEQ ID NO:2 and SEQ ID NO:3). Provided that the nucleotide sequence retains the property of silencing expression of a lycopene epsilon cyclase (ε-CYC) gene or sequence,

Furthermore, the present invention provides a method for altering the carotenoid profile in a plant or a tissue within the plant comprising,

i) providing the plant comprising:

a) a first nucleic acid sequence comprising a regulatory region operatively associated with a silencing nucleotide sequence, wherein expression of the silencing nucleotide sequence reduces or eliminates the expression of a lycopene epsilon cyclase, and
b) one or more than one second nucleic acid sequence, wherein each of the one or more than one second nucleic acid sequence comprise a regulatory region operatively associated with a sequence that encodes one or more than one enzyme involved in carotenoid synthesis, and

ii) expressing the silencing nucleotide sequence and the one or more than one second nucleic acid sequence within the plant or a tissue within the plant, wherein expression of the silencing nucleotide sequence reduce the level of the lycopene epsilon cyclase in the plant or within a tissue of the plant, the reduced level of lycopene epsilon cyclase may be determined by comparing the level of expression of the lycopene epsilon cyclase in the plant, or a tissue of the plant, with a level of the lycopene epsilon cyclase in a second plant, or the tissue from the second plant, that does not express the silencing nucleic acid sequence, and expression of the one or more than one second nucleic acid sequence results in increased expression of a the one or more than one enzyme involved in carotenoid synthesis.

Examples of one or more than one additional nucleotide sequence that may be coexpressed in a plant as outlined above include, but are not limited to beta carotene hydroxylase (Yu et al. 2007, which is incorporated herein by reference), beta carotene 3-hydroxylase (Cunningham and Gantt, 2005, which is incorporated herein by reference), beta carotene ketolase (Cunningham and Gantt, 2005, which is incorporated herein by reference), phytoene synthase (Misawa et al. 1994, which is incorporated herein by reference), phytoene desaturase (Bartley et al 1999, which is incorporated herein by reference), zeaxanthin epoxidase (Latowski et al, 2007, which is incorporated herein by reference).

A plant comprising the first nucleic acid sequence as defined above, may be crossed, using standard methods known to one of skill in the art, with a plant comprising the one or more than one second nucleic acid sequence as defined above so that the progeny express both the first nucleic acid sequence and the one or more than one second nucleic acid sequence. Alternatively, A plant comprising the first nucleic acid sequence as defined above, may be transformed, using standard methods known to one of skill in the art or as described herein, with a construct comprising the one or more than one second nucleic acid sequence as defined above, or a plant comprising the one or more than one second nucleic acid sequence as defined above, may be transformed, using standard methods known to one of skill in the art or as described herein, with a construct comprising the first nucleic acid sequence as defined above, in order to produce a plant that expresses both the first nucleic acid sequence and the one or more than one second nucleic acid sequence.

Plants may comprise combinations of nucleic acid sequences. These sequences may be introduced into a plant using standard techniques, for example, but not limited to, by introducing one or more than one nucleic acid into a plant by transformation, or by introducing one, two, or more than two, silencing nucleic acid sequences, each silencing nucleic acid sequence comprising a sequence directed against a target gene, into a plant by transformation. Alternatively, silencing nucleic acid sequences may be introduced into a plant by crossing a first plant with a second plant that comprises one or more than one first gene fusion, or by crossing a first plant comprising one or more than one first gene fusion with a second plant comprising one or more than one second gene fusion. Silencing nucleic acid sequences may also be introduced into a plant by crossing a first plant with a second plant that comprises one, two, or more than two, silencing nucleic acid sequences. Each silencing nucleic acid sequence may comprise a sequence directed at silencing a lycopene epsilon cyclase (ε-CYC), or a portion of the lycopene epsilon cyclase.

Furthermore, analogues of any of the silencing nucleotide sequences encoding the lycopene epsilon cyclase (ε-CYC) may be used according to the present invention. An “analogue” or “derivative” includes any substitution, deletion, or addition to the silencing nucleotide sequence, provided that the nucleotide sequence retains the property of silencing expression of a lycopene epsilon cyclase (ε-CYC) gene or sequence, reducing expression of a lycopene epsilon cyclase sequence, or reducing synthesis or activity of a protein encoded by the lycopene cyclase (ε-CYC) sequence. For example, derivatives, and analogues of nucleic acid sequences typically exhibit greater than 80% similarity with, a silencing nucleic acid sequence. Sequence similarity, may be determined by use of the BLAST algorithm (GenBank: www.ncbi.nlm.nih.gov/cgi-bin/BLAST/), using default parameters (Program: blastn; Database: nr; Expect 10; filter: low complexity; Alignment: pairwise; Word size: 11). Analogs, or derivatives thereof, also include those nucleotide sequences that hybridize under stringent hybridization conditions (see Maniatis et al., in Molecular Cloning (A Laboratory Manual), Cold Spring Harbor Laboratory, 1982, p. 387-389, which is incorporated herein by reference) to any one of the sequences described herein, provided that the sequences exhibit the property of silencing expression of a lycopene epsilon cyclase (ε-CYC) gene. For example, wherein the silencing nucleotide sequence exhibits reduces expression of a lycopene epsilon cyclase (8-CYC) gene or sequence from about 10 to about 100%. An example of one such stringent hybridization conditions may be hybridization with a suitable probe, for example but not limited to, a [γ-³²P]dATP labelled probe for 16-20 hrs at 65° C. in 7% SDS, 1 mM EDTA, 0.5M Na₂HPO₄, pH 7.2. Followed by washing in 5% SDS, 1 mM EDTA 40 mM Na₂HPO₄, pH 7.2 for 30 min at 65° C., followed by washing in 1% SDS, 1 mM EDTA 40 mM Na₂HPO₄, pH 7.2 for 30 min at 65° C. Washing in this buffer may be repeated to reduce background. An alternate example of stringent hybridization involves, hybridization in Church buffer (Church and Gilbert 1984, which is incorporated herein by reference) at 61° C. for 22 h, washing the filter twice in 2×SSC, 0.1% SDS for 10 min at 61° C., and washing twice in 0.2×SSC, 0.1% SDS for 10 min at 61° C.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a gene of interest, this may result in expression of the gene of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element comprises a basal promoter element, responsible for the initiation of transcription, as well as other regulatory elements (as listed above) that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see—specific a regulatory region, include the napin promoter, and the cruciferin promoter (Rask et al., 1998, J. Plant Physiol. 152: 595-599; Bilodeau et al., 1994, Plant Cell 14: 125-130, each of which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I.R.P., 1998, Trends Plant Sci. 3, 352-358; which is incorporated by reference). Examples, of potential inducible promoters include, but not limited to, tetracycline-inducible promoter (Gatz, C., 1997, Ann. Rev. Plant Physiol. Plant Mol. Biol. 48, 89-108; which is incorporated by reference), steroid inducible promoter (Aoyama, T. and Chua, N.H., 1997, Plant J. 2, 397-404; which is incorporated by reference) and ethanol-inducible promoter (Salter, M. G., et al, 1998, Plant Journal 16, 127-132; Caddick, M. X., et al, 1998, Nature Biotech. 16, 177-180, which are incorporated by reference) cytokinin inducible IB6 and CKI1 genes (Brandstatter, I. and Kieber, J. J., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

A constitutive regulatory region directs the expression of a gene throughout the various parts of a plant and continuously throughout plant development. Examples of known constitutive regulatory elements include promoters associated with the CaMV 35S transcript. (Odell et al., 1985, Nature, 313: 810-812), the rice actin 1 (Zhang et al, 1991, Plant Cell, 3: 1155-1165), actin 2 (An et al., 1996, Plant J., 10: 107-121), or tms 2 (U.S. Pat. No. 5,428,147, which is incorporated herein by reference), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), the tobacco translational initiation factor 4A gene (Mandel et al, 1995 Plant Mol. Biol. 29: 995-1004), and tCUP (WO 99/67389, which is incorporated herein by reference). The term “constitutive” as used herein does not necessarily indicate that a gene under control of the constitutive regulatory region is expressed at the same level in all cell types, but that the gene is expressed in a wide range of cell types even though variation in abundance is often observed.

The silencing nucleotide sequence may be expressed in any suitable plant host that is transformed by the nucleotide sequence, or constructs, or vectors of the present invention. Examples of suitable hosts include, but are not limited to, agricultural crops including canola, Brassica spp., maize, tobacco, alfalfa, potato, ginseng, pea, oat, rice, soybean, wheat, barley, sunflower, and cotton. Any member of the Brassica family can be transformed with one or more genetic constructs of the present invention including, but not limited to, canola, Brassica napus, B. carinata, B. nigra, B. oleracea, B. chinensis, B. cretica, B. incana, B. insularis, B. japonica, B. atlantica, B. bourgeaui, B.narinosa, B. juncea, B. rapa, Arabidopsis thaliana.

The one or more chimeric genetic constructs of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. One or more of the chimeric genetic constructs of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence.

Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes and the small subunit of the ribulose-1,5-bisphosphate carboxylase (ssRUBISCO) gene.

To aid in identification of transformed plant cells, the constructs of this invention may be further manipulated to include plant selectable markers. Useful selectable markers include enzymes that provide for resistance to chemicals such as an antibiotic for example, gentamycin, hygromycin, kanamycin, or herbicides such as phosphinothricin, glyphosate, chlorosulfuron, and the like. Similarly, enzymes providing for production of a compound identifiable by colour change such as GUS (beta-glucuronidase), or luminescence, such as luciferase or GFP, may be used.

Also considered part of this invention are transgenic plants containing the chimeric gene construct of the present invention. Methods of regenerating whole plants from plant cells are also known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue cultures.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); Miki and Iyer, Fundamentals of Gene Transfer in Plants. in Plant Metabolism, 2d Ed. DT. Dennis, D H Turpin, D D Lefebrve, D B Layzell (eds), Addison Wesly, Langmans Ltd. London, pp. 561-579, 1997), or Clough and Bent, (1998, Plant J. 16, 735-743), and Moloney et al. (1989, Plant Cell Rep. 8, 238-242).

As demonstrated in the example below, enhanced levels of carotenoids, including β-carotene, lutein and violaxanthin, zeaxanthin and beta-cryptoxanthin (see Example 3, Table 3), were obtained in the seed of B. napus plants, following the selective downregulation of the expression of ε-CYC. Furthermore, transgenic seeds exhibited slight reductions in lipid content and minor alterations in fatty acid profiles relative to the wild type control (see Example 3, Table 4). These seeds may therefore be used to obtain canola quality oil, while at the same time be used to obtain increased levels of carotenoids.

B. napus lines with elevated concentrations of astaxanthin in the seed were produced. This was achieved by cloning two genes for astaxanthin biosynthesis from the petals of Adonis aestivalis and inserting them into B. napus (Example 4). Several B. napus lines were developed that contained the genes responsible for astaxanthin synthesis and many of these lines had increased concentrations of astaxanthin in the seeds (Example 4, Tables 6-8). B. napus lines were also modified to produce high concentrations of the astaxanthin precursor β-carotene.

The carotenoids may be extracted from the seed using standard techniques as known to one of skill in the art, and further purified using HPLC or other chromatographic or separation techniques as are known in the art to obtain one or more than one of the desired compound, for example, but not limited to 3-carotene, lutein and violaxanthin, zeaxanthin and beta-cryptoxanthin. For example, the carotenoids may be purified by pulverizing seed with a extraction solvent, for example but not limited to hexane/acetone/ethanol, followed by centrifugation, collecting the supernatant, and concentrating the fraction by removing the extraction solvent, for example by evaporation. Triacyl glycerides may be saponified using methanolic-KOH, and carotenoids and any aqueous compounds partitioned using for example water-petroleum ether. The ether phase may be concentrated by evaporation, and the sample prepared for HPLC separation. A non limiting example of HPLC separation may involve, resuspending the sample in a suitable mobile phase, for example, acetonitrile/methylene chloride/methanol with butylated hydroxytoluene followed by analysis using HPLC-PDA, using an appropriate column, for example a YMC “Carotenoid Column” reverse-phase C₃₀, 5 μm column (Waters Ltd, Mississauga, ON, Canada) and comparing the elution of compounds with those of known standards. However, other methods that are known to one of skill in the art may also be used, and the invention is not limited to methods of extracting carotenoids or fatty acids from seed.

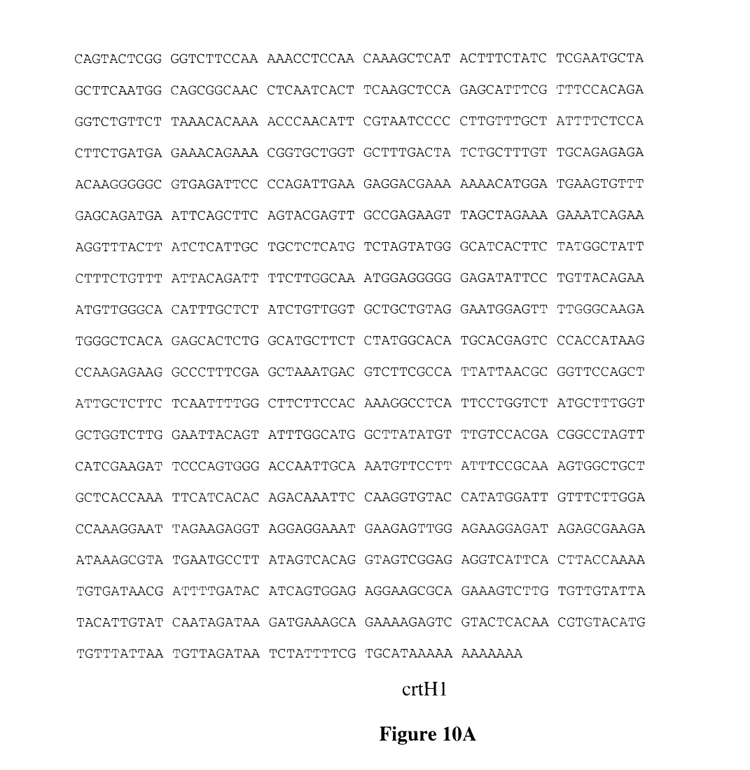

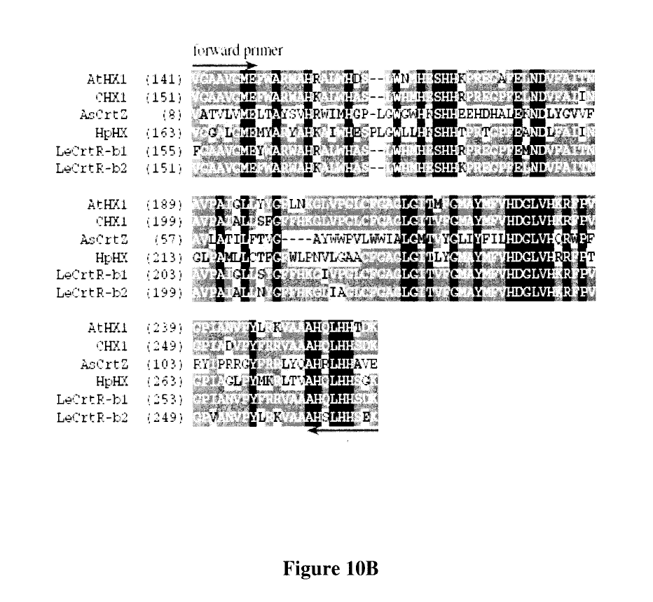

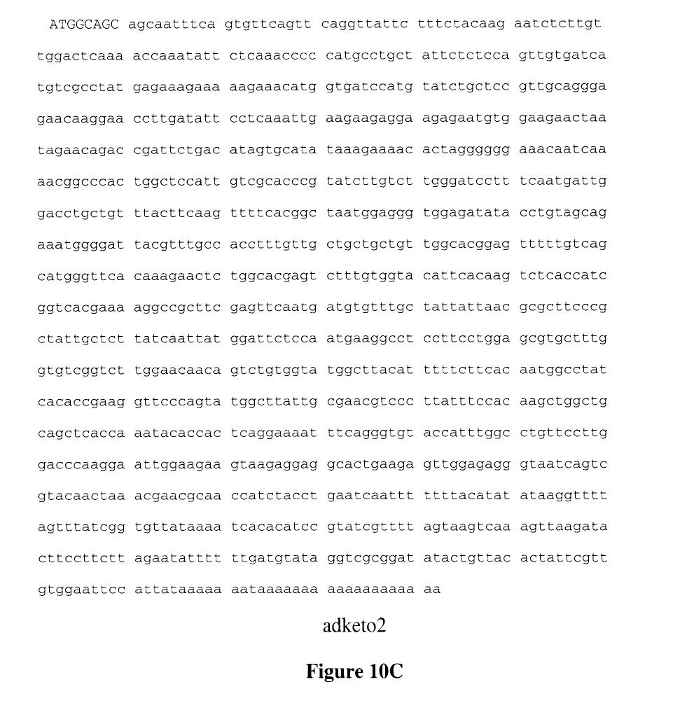

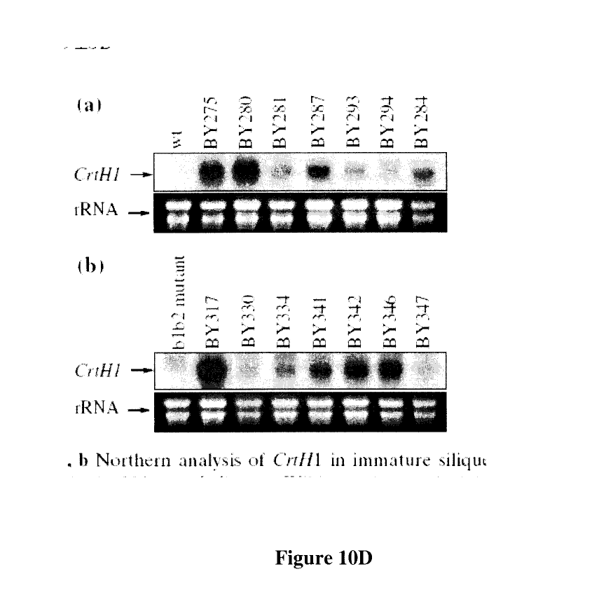

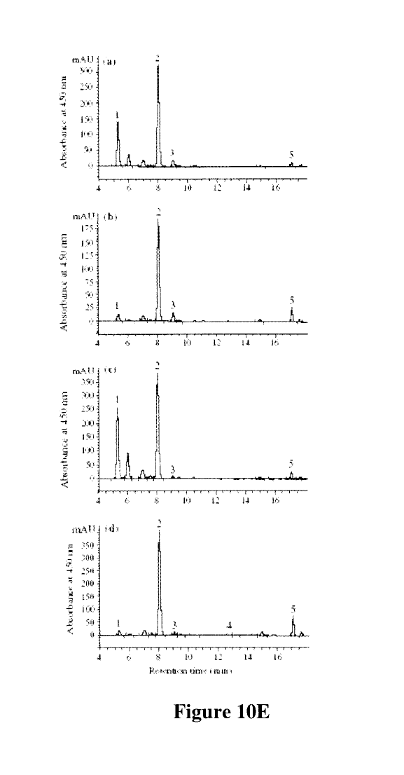

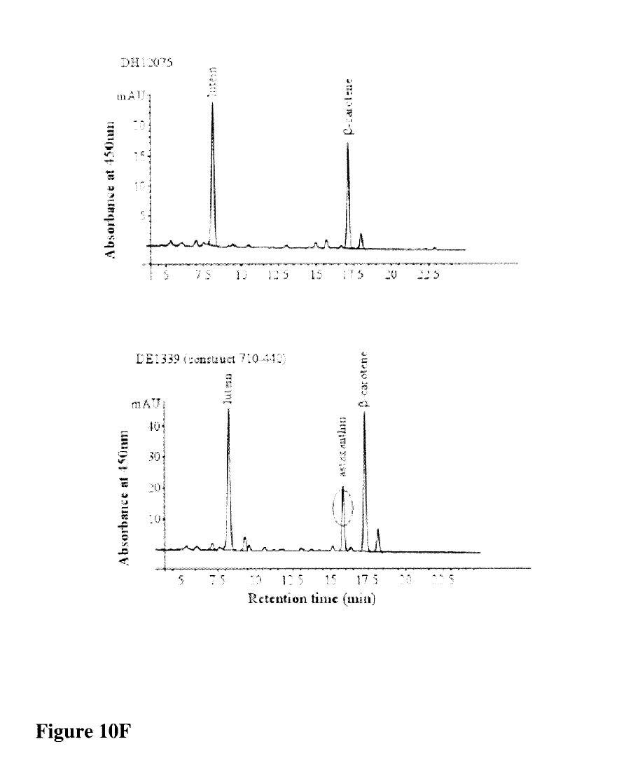

Sequences listed in Application:

| SEQ ID NO | Sequence description | Figure Ref: |
|---|---|---|
| SEQ ID NO: 1 | Brassica napus lycopene epsilon cyclase sequence | FIG. 2D |
| SEQ ID NO: 2 | Brassica napus lycopene epsilon cyclase cDNA | FIG. 2B |
| | 5′-end with 16 nt trailer sequence on each end | |
| SEQ ID NO: 3 | Brassica napus lycopene epsilon cyclase cDNA | FIG. 2C |
| | 3′-end with 16 nt trailer sequence on each end | |
| SEQ ID NO: 4 | A. thaliana lycopene ε-cyclase (ε-CYC; | FIG. 2A |
| | NM_125085) | |
| SEQ ID NO: 5 | Primer 1; 5′end specific RNAi construct 352 bp | |
| SEQ ID NO: 6 | Primer 2; 5′end specific RNAi construct 352 bp | |
| SEQ ID NO: 7 | Primer 3; 3′end specific RNAi construct 410 bp | |
| SEQ ID NO: 8 | Primer 4; 3′end specific RNAi construct 410 bp | |
| SEQ ID NO: 9 | Primer 5; PCR determination constructs 710-422 | |
| | and 710-423 | |
| SEQ ID NO: 10 | Primer 6; (384bp ε-CYC - specific fragment used | |
| | as probe when used with P7) | |
| SEQ ID NO: 11 | Primer 7; (384bp ε-CYC - specific fragment used | |
| | as probe when used with P6) | |
| SEQ ID NO: 12 | Primer 8; PSY (phytoene synthase, 803 bp) | |
| SEQ ID NO: 13 | Primer 9; PSY (phytoene synthase, 803 bp) | |
| SEQ ID NO: 14 | Primer 10; PDS (phytoene desaturase, 454 bp) | |
| SEQ ID NO: 15 | Primer 11; PDS (phytoene desaturase, 454 bp) | |
| SEQ ID NO: 16 | Primer 12; β-cyc (lycopene β-cyclase, 438 bp) | |
| SEQ ID NO: 17 | Primer 13; β-cyc (lycopene β-cyclase, 438 bp) | |
| SEQ ID NO: 18 | Primer 14; ε-cyc (lycopene ε-cyclase, 418 bp) | |
| SEQ ID NO: 19 | Primer 15; ε-cyc (lycopene ε-cyclase, 418 bp) | |
| SEQ ID NO: 20 | Primer 16; ε-cyc (1.8 kbp) | |
| SEQ ID NO: 21 | Primer 17; ε-cyc (1.8 kbp) | |
| SEQ ID NO: 22 | Primer 18; ACT (actin, 700 bp) | |
| SEQ ID NO: 23 | Primer 19; ACT (actin, 700 bp) | |
| SEQ ID NO: 24 | Primer 20; ACT (actin, 1178 bp) | |
| SEQ ID NO: 25 | Primer 21; ACT (actin, 1178 bp) | |
| SEQ ID NO: 26 | Forward primer; CrtH1 ORF A. aestivalis | |
| SEQ ID NO: 27 | Reverse primer; CrtH1 ORF A. aestivalis | |
| SEQ ID NO: 28 | β-CYC (beta CYC; NM_111858) A. thaliana | FIG. 2A |
| SEQ ID NO: 29 | Forward primer; Adketo2 ORF | |
| SEQ ID NO: 30 | Reverse primer; Adketo2 ORF | |
| SEQ ID NO: 31 | β-CYC - 5′ of B. napus | FIG. 2E |
| SEQ ID NO: 32 | β-CYC - 3′ of B. napus | FIG. 2F |
| SEQ ID NO. 33 | β-CYC - mid of B. napus | FIG. 2G |
| SEQ ID NO 34 | Epsilon CYC -mid of B. napus | FIG. 2G |
| SEQ ID NO 35 | 5′ end of eps cyc of B. napus | FIG. 2E |
| SEQ ID NO 36 | 3′ end of eps cyc of B. napus | FIG. 2F |
| SEQ ID NO: 37 | crtH1 (from Adonis aestivalis) | FIG. 10A |
| SEQ ID NO 38 | adketo2 (from Adonis aestivalis) | FIG. 10B |

### EXAMPLES

### Example 1

### Vectors, and Methods

### Vector Construction for RNAi and Plant Transformation

Two B. napus ESTs, EST CL1624 and EST CL1622 homologous to the 5′- and 3′-ends, respectively, of the A. thaliana lycopene ε-cyclase (ε-CYC; NM_{—}125085; SEQ ID NO: 4) were identified from a B. napus EST collection held at the Saskatoon Research Centre (see the following URL—brassica.ca). These two ESTs were used to generate RNAi constructs specific to the 5′ and 3′ ends of ε-CYC (SEQ ID NO:2 and SEQ ID NO:3; FIG. 2). Primers with built-in SpeI and AscI or BamHI and SwaI sites (Table 1) were used to produce the 352 by of 5′-end (primer P1, SEQ ID NO:5; and P2; SEQ ID NO:6) and 410 by of 3′-end (primer P3; SEQ ID NO:7; and P4) gene products by PCR amplification.

Primers:

(SEQ ID NO: 5)

P1-5′-cgactagtggcgcgccGAGGTTTTCGTCTCCG-3′;
restriction sites of SpeI and AscI indicated with
lower case;

(SEQ ID NO: 6)

P2-5′-cgggatccatttaaatCATCCATGTCTTTGTTCTG-3′;
restriction sites of BamHI and Swal indicated with
lower case;

(SEQ ID NO: 7)

P3-5′-cgactagtggcgcgccCAGAAAGGAAACGACAA-3′;
restriction sites of SpeI and AscI indicated with
lower case;

(SEQ ID NO: 8)
P4-5′-cgggatccatttaaatCAATCTTCTAAGGCACGC-3′;

restriction sites of BamHI and SwaI indicated with
lower case

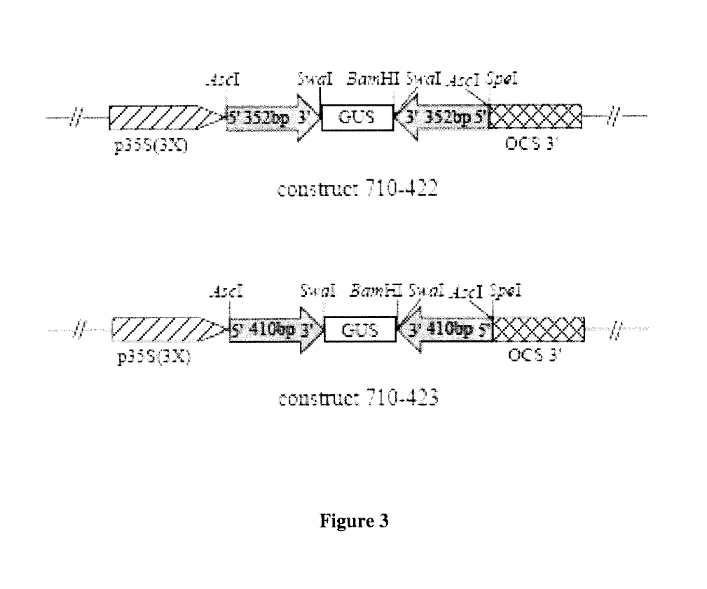

Single palindromic repeats of the 5′ and 3′-end PCR products were inserted around a 300 bp spacer of β-glucuronidase in pGSA1285 vector (CAMBIA, Canberra, ACT, Australia). The resulting RNAi vectors were designated 710-422 for the 5′-end fragment or 710-423 for the 3′-end fragment (see FIG. 3).

### Growth Conditions and Plant Transformation

B. napus plants were grown in soil-less mix according to the protocol described by Stringham (1971, which is incorporated herein by reference) in a controlled environment greenhouse (16 hr light/8 hr dark, 20° C./17° C.).

Cotyledon explants of B. napus DH12075 were used for transformation mediated by Agrobacterium tumefaciens GV3101PVP90 according to the method by Moloney et al (1989, which is incorporated herein by reference). Only those plants shown to be transgenic determined by PCR were subjected to further analysis. The primers used for this PCR determination are P5 (SEQ ID NO:9) and P2 (SEQ ID NO:6) for construct 710-422, P5 (SEQ ID NO:9) and P4 (SEQ ID NO:8) for construct 710-423.

### DNA Isolation and Southern Blot Analysis

Total genomic DNA was isolated from leaves of B. napus using DNeasy Plant Mini Kit (Qiagen, Mississauga, Canada). Approximately 10 μg of genomic DNA was digested with BamHI, EcoRI, EcoRV, SalI, SpeI and SstI and separated on a 0.8% agarose gel, transferred onto Hybond-XL membrane (Amersham Biosciences, Quebec, Canada) and hybridized with lycopene ε-cyclase-specific fragment labeled with [α-³²P]dCTP using random primers. The probe was purified with ProbeQuant G-50 Micro Column (Amersham Biosciences, QC, Canada). The 384 bp ε-CYC-specific fragment (nucleotides 75-427 of SEQ ID NO:1) used as probe was amplified by PCR using primers P6 and P7. The PCR product was isolated from 1.0% agarose gel and purified with QIAquick Gel Extraction Kit (Qiagen, Mississauga, Canada). Hybridization was performed with Church buffer (Church and Gilbert 1984, which is incorporated herein by reference) at 61° C. for 22 h. The filter was washed twice in 2×SSC, 0.1% SDS for 10 min at 61° C. and followed by washing twice in 0.2×SSC, 0.1% SDS for 10 min at 61° C. The filter was then exposed to an X-ray film with an intensifying screen at −70° C. for 7 days.

### RNA Isolation and Semi-Quantitative RT-PCR

Total RNA was isolated from leaves, flower petals, roots and seeds at different developmental stages as described by Carpenter et al. (1998) with some modifications and used for one-step, semi-quantificative Reverse Transcriptase Polymerase Chain Reaction (RT-PCR) analysis of PSY, phytoene synthase; PDS, phytoene desaturase; beta-CYC, lycopene, beta-cyclase; epsilon-CYC, lycopene epsilon-cyclase gene expression. This method was chosen because the low abundance of many carotenoid biosynthetic gene steady state mRNAs (Giuliano et al. 1993).

About 100 mg of ground tissue was extracted with 600 ml of RNA extraction buffer (0.2M Tris-HCl, pH 9.0, 0.4M LiCl, 25 mM EDTA, 1% SDS) and an equal volume of Tris-HCl buffered phenol (pH 7.9). Extraction was repeated twice with phenol and followed once with chloroform. Approximately ¼ volume of 10 M LiCl was added to the decanted aqueous layer, mixed well, stored at 4° C. overnight and then centrifuged at 14,000 g for 20 min. The pellet was resuspended in 0.3 ml of DEPC-treated dH₂O, to which 30 μl of 3M sodium acetate, pH 5.3 and 0.7 ml of 95% ethanol were added. The mixture was chilled at −70° C. for 10 min and then centrifuged at 14 000 g for 20 min. The pellet was washed and resuspended in 20 μl of DEPC-treated dH₂O.

For semi-quantitative RT-PCR, total RNA was treated with Amplification Grade DNase I (Invitrogen, Burlington, ON, Canada) according to the manufacture's instructions. RT-PCR co-amplification of an internal standard actin gene and test gene fragments were performed using 180 ng of total RNA and 25 μl of the SuperScript™ One-Step RT-PCR Kit (Invitrogen). Reverse transcription was performed at 45° C. for 30 min, followed by PCR amplification using an initial denaturation at 94° C. for 4 min, then 26 cycles at 94° C. (30 sec), 55° C. (30 sec), 72° C. (50 sec) and a final extension at 72° C. for 5 min.

Primers spanning introns were designed for each gene, except β-CYC which does not have an intron, to distinguish between products resulting from amplification of cDNA and genomic DNA. For analysis of ε-CYC and PDS gene expression, a 1178 by actin internal control was used. For the other genes a 700 bp actin was used. 1.8 Kbp ε-CYC was used for analysis of ε-CYC in developing seeds of transgenic lines. The following primer combinations were used in the RT-PCR reaction (see Table 1):

P8 and P9 for PSY (phytoene synthase, 803 bp);

P10 and P11 for PDS (phytoene desaturase, 454 bp);

P12 and P13 for β-cyc (lycopene β-cyclase, 438 bp);

P14 and P15 for s-cyc (lycopene s-cyclase, 418 bp);

P16 and P17 for ε-cyc (1.8 Kbp);

P18 and P19 for ACT-(actin, 700 bp);

P20 and P21 for ACT-(actin, 1178 bp).

| | | SEQ ID |
|---|---|---|
| Primer | Primer Sequence | NO: |
| P1 | 5′-cgactagtggcgcgccGAGGTTTTCGTCTCC | 5 |
| | G-3′ | |
| P2 | 5′-cgggatccatttaaatCATCCATGTCTTTGTTCT | 6 |
| | G-3′ | |
| P3 | 5′-cgactagtggcgcgccCAGAAAGGAAACGACA | 7 |
| | A-3′ | |
| P4 | 5′-cgggatccatttaaatCAATCTTCTAAGGCACG | 8 |
| | C-3′ | |
| P5 | 5′-CAATCCCACTATCCTTCGCAAGACCC-3′ | 9 |
| P6 | 5′-GAGGTTTTCGTCTCCG-3′ | 10 |
| P7 | 5′-CATCCATGTCTTTGTTCTG-3′ | 11 |
| P8 | 5′-CTTAGACAAGCGGCTTTGGTGAACA-3′ | 12 |
| P9 | 5′-ACTGAGCTCGGTGACGCCTTTCTC-3′ | 13 |
| P10 | 5′-CGAGATGCTGACATGGCCAGAGAAA-3′ | 14 |
| P11 | 5′-AGTGGCAAACACATAAGCGTCTCCT-3′ | 15 |
| P12 | 5′-AGAGAGGATGGTTGCGAGGCTGAA-3′ | 16 |
| P13 | 5′-AACAGCCTCGACGACAAGAAACCA-3′ | 17 |
| P14 | 5′-CACCAGAAAGGAAACGACAAAGAGCA-3′ | 18 |
| P15 | 5′-TTCTCTCAATCTTCTAAGGCACGCAC-3′ | 19 |
| P16 | 5′-GAGGTTTTCGTCTCCG-3′ | 20 |
| P17 | 5′-TTCTCTCAATCTTCTAAGGCACGCAC-3′ | 21 |
| P18 | 5′-TGAAAGATGGCCGATGGTGAGGA-3′ | 22 |
| P19 | 5′-CCGTCTCCAGCTCTTGCTCGTAGT-3′ | 23 |
| P20 | 5′-TGAAAGATGGCCGATGGTGAGGA-3′ | 24 |
| P21 | 5′-CACACTCACCACCACGAACCAGAAG-3′ | 25 |

The RT-PCR products were separated on 1.0% agarose gel and transferred to Hybond-XL membrane (Amersham Biosciences, QC, Canada). The blots were probed with [α-³²P]dCTP labeled gene-specific fragment. EtBr-stained gel photograph was used for internal control gene actin.

### Microarray Analysis of Carotenoid Gene Expression

Ambion AminoAllyl MessageAmp II aRNA amplification kit was used for aRNA amplification and labelling according to the manufacture's instructions (Austin, Tex. USA). CyDye Post-labelling reactive dye pack was purchased from Amersham (GE healthcare, Baie d'Urfe, QC, Canada). Initial data processing and analysis were performed in BASE database (see the following URL: base.thep.lu.se). B. napus 15K oligo arrays were used.

Extraction of Carotenoids from B. napus Seeds and HPLC Analysis

Approximately 200 mg of seed with 3 ml of hexane/acetone/ethanol (50/25/25) extraction solvent was pulverized by rapidly shaking for 30 min in a scintillation vial containing a steel rod (adapted from Shewmaker et al. 1999). The sample was centrifuged for 10 minutes at 1,800 g and the supernatant collected. The pellet was washed with another 3 ml extraction solvent and the supernatant collected and pooled. The solvent was removed by evaporation at room temperature under a stream of nitrogen gas. Triacyl glycerides were saponified in the residue by heating at 80° C. for 1 hour in 5 ml methanolic-KOH (10% w/v KOH in methanol:water [80:20 v/v]). Carotenoids and aqueous compounds were partitioned using 2 ml H₂O and 3 ml petroleum ether. The ether phase and a single 3 ml wash were collected, pooled and the solvent was evaporated at room temperature under a nitrogen gas stream. The residue was resuspended in 200 μl of acetonitrile/methylene chloride/methanol (50/40/10 [v/v]) with 0.5% (w/v) butylated hydroxytoluene (BHT) and filtered through a 0.2 μm pore size 4 mm nylon syringe filter into an HPLC sample vial. The extract was immediately analysed using HPLC-PDA. Aliquots of 20 μl were loaded onto a YMC “Carotenoid Column” reverse-phase C₃₀, 5 μm column (4.6×250 mm) (Waters Ltd, Mississauga, ON, Canada) at 35° C. Mobile phases consisted of methanol (A) and tert-methyl butyl ether (B). The gradient elution used with this column started at 95% A and 5% B, and then followed by a linear gradient to 35% A and 65% B in 25 min. A flow rate of 1.2 ml/min was used, and the eluate was monitored at 450 nm. Peaks were identified by their retention time and absorption spectra compared to those of known standards. Quantification of carotenoids was conducted using curves constructed with authentic standards.

### Fatty Acid Analysis

The gas chromatography method described by Young et al (2006) was used to determine fatty acid concentration and profile. Briefly, triplicate samples of approximately 30 mg of seed were homogenised in hexane containing 0.938 mg/ml heptadecanoic acid methyl ester (HAME; Sigma-Aldrich, Oakville, ON, Canada) as an internal standard. Lipids were transesterified in 6.7% sodium methoxide for 30 minutes and the solution neutralised in 10% citric acid. The hexane layer was filtered through a 45 μm PTFE syringe filter and a 1:20 dilution made. One microlitre of diluted methyl ester solution was injected in a DBwax column (10 m long, 0.1 mm ID, 0.2 μm film, Agilent Technologies Canada, Mississauga, ON, Canada) in a Hewlet Packard 6890 CG. Inlet temperature was set at 240° C., with hydrogen carrier gas and a 1/20 split, using nitrogen makeup gas. Column temperatures started at 150°, ramped to 220° at 50° C./min and were maintained for seven minutes. Column pressure started at 50 psi at insertion and dropped to approximately 35 psi after two minutes. Fatty acid methyl esters were detected using a flame ionisation detector.

### Example 2

### Carotenoid Profile in Leaves, Petals and Developing Seeds of B. napus

The carotenoid accumulation profile in B. napus (DH12075; non-transformed) leaves, petals and developing seeds were determined using HPLC analysis. In leaves lutein, β-carotene, violaxanthin and β-cryptoxanthin account for 43.30%±1.21, 44.16%±5.63 11.46%±0.75 and 0.84±0.05 of total carotenoids, respectively (Table 2).

TABLE 2
Carotenoid profile in B. napus (DH12075)

| | | | Violaxanthin | Zeaxanthin | β-Cryptoxanthin |
|---|---|---|---|---|---|
| Tissue | B-Carotene μg/g FW | Lutein μg/g FW | μg/g FW | μg/g FW | μg/g FW |
| Leaf | 106.91 ± 13.63 | 104.83 ± 2.92 | 27.74 ± 1.81 | 0.59 ± 0.02 | 2.03 ± 0.11 |
| Petal | 6.74 ± 0.51 | 21.75 ± 0.92 | 14.84 ± 1.25 | 1.23 ± 0.17 | 4.33 ± 0.27 |
| 15-20 DPA seed | 6.30 ± 0.22 | 7.47 ± 0.25 | 3.30 ± 0.24 | 0.09 ± 0.005 | 0.13 ± 0.004 |
| 35-40DPA seed | 19.23 ± 0.71 | 21.22 ± 0.30 | 2.09 ± 0.15 | 0.26 ± 0.04 | UD |
| fresh mature seed | 0.06 ± 0.005 | 3.14 ± 0.26 | 0.09 ± 0.01 | UD | UD |
| dry mature seed | 0.49 ± 0.28 | 3.30 ± 0.49 | UD | UD | UD |

UD, undetectable;
FW, fresh weight;
DPA, days post-anthesis;
Each value is the mean result from triplicate ± SD.

The levels of violaxanthin (30.34%±2.55) and β-cryptoxanthin (8.85%±0.55) in the petals were higher than in the leaves, but the level of β-carotene (13.79%±1.04%) was lower. The profiles of carotenoids accumulating in the seed varied depending on the developmental stage (Table 2). The highest level of violaxanthin was detected in seeds 15-20 days post anthesis (DPA), and then gradually decreased as the seed matured. Seeds at 35-40 DPA had the highest levels of lutein and β-carotene, followed by a sharp drop in fresh mature- and dry mature-seeds. Trace amounts of zeaxanthin were detected in seeds at 15-20 DPA to 35-40 DPA, but it was undetectable in fresh mature- and dry mature-seeds. β-cryptoxanthin, which is rapidly converted to zeaxanthin, was only detectable in seeds at 15-20 DPA.

### Expression Profiles of Genes in the Carotenoid Biosynthesis Pathway

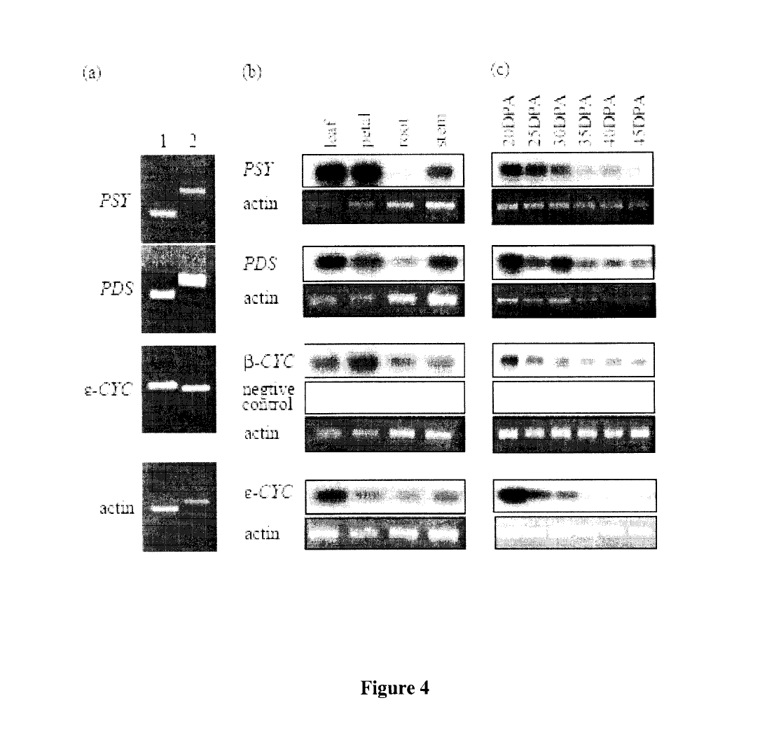

Semi-quantitative RT-PCR analysis was used to determine whether a correlation exists between carotenoid profiles and transcript abundance of some carotenoid biosynthesis genes (FIGS. 4a-c). Primers spanning intron regions were designed for each gene, except for the intron-free β-CYC, to allow PCR products amplified from residual genomic DNA and target cDNA to be distinguished.

RT-PCR analysis revealed that PSY, PDS, (3-CYC and ε-CYC (PSY, phytoene synthase; PDS, phytoene desaturase; beta-CYC, lycopene, beta-cyclase; epsilon-CYC, lycopene epsilon-cyclase) were highly expressed in leaves, petals and stems with relatively weaker expression in roots (FIG. 4b). During seed development, the expression of PSY and ε-CYC was highest in early stages, i.e. up to 30 DPA, but decreased afterwards. In contrast, expression of PDS and β-cyc was almost unchanged during seed development (FIG. 4c).

### Example 3

### Silencing of ε-CYC Increased Levels of β-Carotene and Lutein

Two RNAi constructs, 710-422 and 710-423 (FIG. 3), were made to the 5′ and 3′ ends of B. napus ε-CYC and transformed into B. napus DH12075. Transgenic plants were subjected to RT-PCR analysis to determine the expression levels of ε-CYC and the other carotenoid biosynthesis genes, namely PSY, PDS and β-CYC (PSY, phytoene synthase; PDS, phytoene desaturase; beta-CYC, lycopene beta cyclase). As shown in FIG. 5, only the expression of ε-CYC was reduced in transgenic lines relative to the untransformed wild type control DH12075. Expression of the other genes was mostly unaffected.

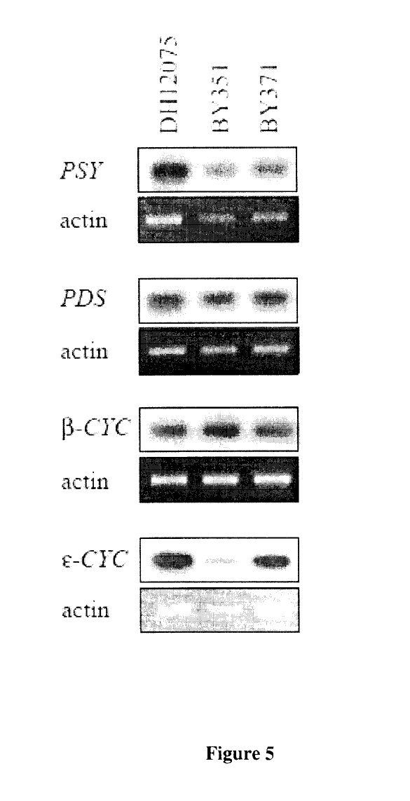

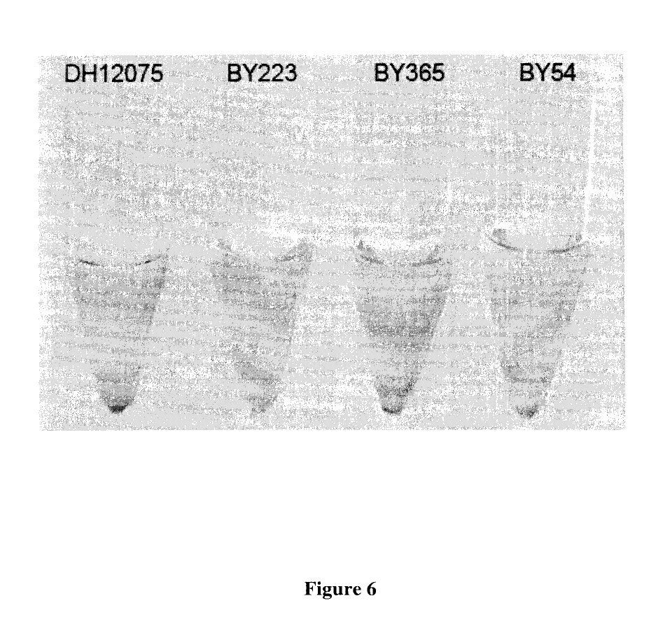

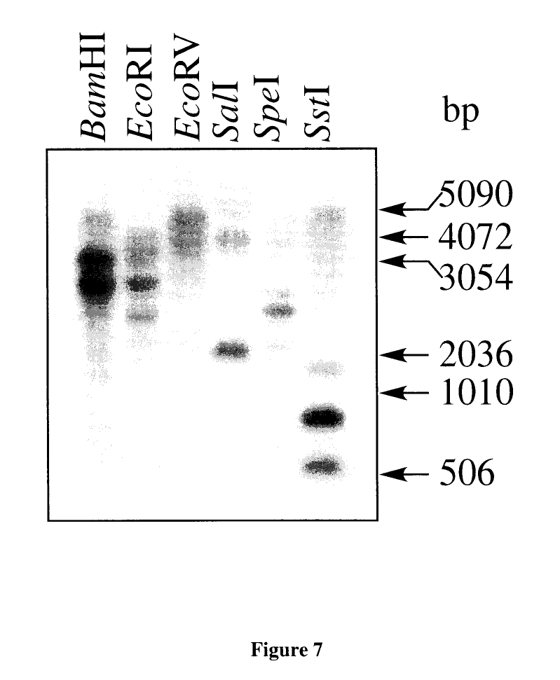

Visual observation of the carotenoid extracts from ε-CYC silenced lines and wild type DH12075 suggested that significant changes in carotenoid content had occurred (FIG. 6). This was confirmed by using HPLC analysis to determine carotenoid profiles in mature seeds of transgenic and DH12075 plants. Seeds of all transgenic lines had enhanced concentrations of total carotenoids, 3.6 to 42.7 times greater than those observed in DH12075. In B. napus DH12075 seeds β-carotene and lutein, both of which are derived from lycopene, were the two main carotenoid compounds present (Table 3).

β-carotene concentrations were at least 6-fold higher in the ε-CYC silenced lines than DH12075, with the greatest amount in line BY269 (185-fold). Lutein concentrations were 3 to 23 fold greater in the transgenic lines. Violaxanthin, zeaxanthin and cryptoxanthin were undetectable in DH12075, but were present in all the transgenic lines with the exception of β-cryptoxanthin in lines BY351, BY58 and BY371. The ratio of β-carotene to lutein approximately doubled in the seeds of most transgenic lines, although 4.8, 4.9 and 8 fold increases in the relative amounts of β-carotene to lutein were observed in BY223, BY365 and BY269, respectively. Interestingly, statistically significant differences in carotenoid profiles were not observed in the leaves of either transgenic or untransformed DH12075 lines (data not shown).

TABLE 3
Concentration of carotenoid compounds in seeds of B. napus expressing epsilon-CYC RNAi (untransformed control
DH12075)

| | | | | | | Total | Ratio of β- |
|---|---|---|---|---|---|---|---|
| | | | Violaxanthin | Zeaxanthin | β-Cryptoxanthin | carotenoid | carotene to |
| Plant line | β-Carotene μg/g FW | Lutein μg/g FW | μg/g FW | μg/g FW | μg/g FW | μg/g FW | lutein |
| DH12075 | 0.49 ± 0.28 | 3.30 ± 0.49 | UD | UD | UD | 5.34 ± 0.96 | 0.15 |
| construct | | | | | | | |
| 710-422 | | | | | | | |
| BY173 | 15.07 ± 4.98 | 39.10 ± 5.33 | 2.43 ± 0.18 | 0.44 ± 0.13 | 0.23 ± 0.04 | 75.48 ± 11.23 | 0.39 |
| BY228 | 19.52 ± 3.27 | 41.74 ± 3.80 | 1.30 ± 0.11 | 2.51 ± 0.69 | 0.08 ± 0.13 | 86.59 ± 8.57 | 0.47 |
| BY241 | 4.18 ± 0.28 | 14.05 ± 0.21 | 0.61 ± 0.02 | 0.21 ± 0.02 | 0.10 ± 0.07 | 24.31 ± 0.56 | 0.30 |
| BY269 | 90.76 ± 6.72 | 76.22 ± 3.74 | 1.74 ± 0.07 | 7.07 ± 0.13 | 0.30 ± 0.13 | 227.78 ± 2.85 | 1.19 |
| BY351 | 6.36 ± 4.14 | 17.44 ± 3.46 | 1.09 ± 0.32 | 0.14 ± 0.03 | UD | 34.44 ± 11.22 | 0.36 |
| construct | | | | | | | |
| 710-423 | | | | | | | |
| BY54 | 11.09 ± 2.06 | 23.15 ± 6.64 | 0.91 ± 0.24 | 0.35 ± 0.03 | 0.16 ± 0.06 | 46.95 ± 8.00 | 0.48 |
| BY58 | 3.34 ± 1.67 | 9.46 ± 3.00 | 0.36 ± 0.13 | 0.33 ± 0.22 | UD | 19.07 ± 5.82 | 0.35 |
| BY223 | 17.88 ± 1.25 | 25.29 ± 1.16 | 1.48 ± 0.12 | 0.31 ± 0.05 | 0.15 ± 0.01 | 59.81 ± 3.19 | 0.71 |
| BY365 | 27.02 ± 4.04 | 37.64 ± 3.95 | 2.42 ± 0.25 | 1.73 ± 0.33 | 0.15 ± 0.03 | 94.09 ± 12.07 | 0.72 |
| BY371 | 3.50 ± 0.31 | 10.36 ± 0.57 | 0.45 ± 0.07 | 0.28 ± 0.01 | UD | 19.15 ± 2.48 | 0.34 |

UD, undetectable;
FW, fresh weight;
Each value is the mean result from triplicate ± SD.

Therefore, therefore the present invention provides a method for increasing the carotenoid contact in a plant by downregulating selectively lycopene epsilon cyclase.

### Microarray Analysis

Microarray analysis was conducted on the developing seeds of line BY351 to investigate the effect of RNAi silencing of ε-CYC on gene expression profiles. Of the 15 000 genes on the array, at least 13 genes were up-regulated by 3-fold compared to DH12075 (Table 4). Only genes with a greater than 3-fold increase in transcript level compared to the parental DH12075 line are listed. Ambion AminoAllyl MessageAmp II aRNA amplification kit was used for RNA amplification and labelling according to the manufacture's instructions (Austin, Tex. USA). CyDye Post-labelling reactive dye pack was purchased from Amersham (GE healthcare, Baie d'Urfe, QC, Canada). Initial data processing and analysis were performed in BASE database (see URL: base.thep.lu.se). B. napus 15K oligo arrays were used.

TABLE 4
Microarray analysis of gene expression
in developing seeds of line BY351

| | Expression | |
|---|---|---|
| | ratio of | Arabidopsis |
| | BY351 to | homolog |
| Up-regulated gene names | DH12075 | locus name |
| hesB-like domain-containing protein similar | 13.09 | At1g10500 |
| to IscA (putative iron-sulfur cluster | | |
| assembly protein) | | |
| invertase/pectin methylesterase inhibitor | 7.28 | At4g25260 |
| family protein | | |
| late embryogenesis abundant protein (M10)/ | 5.21 | At2g41280 |
| LEA protein M10 | | |
| cytochrome b (MTCYB) (COB) | 4.2 | At2g07727 |
| chlorophyll A-B binding protein 165/180 | 4.02 | At1g29920 |
| chloroplast thylakoid lumen protein | 3.8 | At4g02530 |
| armadillo/beta-catenin repeat family | 3.54 | At5g62560 |
| protein/U-box domain-containing protein | | |
| protochlorophyllide reductase B | 3.41 | At4g27440 |
| pathogenesis-related thaumatin family | 3.37 | At1g19320 |
| protein | | |
| meiosis protein-related (DYAD) | 3.14 | At5g51330 |
| leucine-rich repeat transmembrane protein | 3.07 | At2g31880 |
| kinase, putative | | |
| expressed protein similar to myo-inositol | 3.07 | At2g19800 |
| oxygenase | | |
| unknown | 3.06 | At2g12905 |

### Silencing of ε-CYC has Minimal Impact on Fatty Acid Profiles

The fatty acid composition of seeds expressing lycopene epsilon cyclase RNAi was determined. Ten transgenic lines were tested (Table 5). These results demonstrate that the fatty acid profile of the transgenic seed closely resembles the fatty acid profile obtained from the control seed. The overall concentration of fatty acids was lower in eight of the ten plants when compared to DH12075, but the relative levels of the fatty acids remained essentially the same. The amount of palmitic acid in the transgenic seeds increased compared with DH12075, except for BY223 (Table 5). The concentrations of oleic and eicosanoic acid decreased compared with DH12075, except for oleic in BY371. Overall, the magnitude of the changes to the relative concentrations of fatty acids was minor.

TABLE 5
Fatty acid content of seeds of B. napus expressing epsilon-CYC
RNAi (untransformed control: DH12075)

| | | | | | Unknown | | | Unknown 20C | |
|---|---|---|---|---|---|---|---|---|---|
| Plant line | FA content (% FW) | Palmitic | Steric | Oleic | 18C FA | Linoleic | Linolenic | FA | Eicosanoic |
| DH12075 | 35.49 ± 1.08 | 3.58 ± 0.20 | 2.65 ± 0.25 | 62.55 ± 0.83 | 1.84 ± 0.17 | 12.33 ± 0.54 | 6.55 ± 0.34 | 1.05 ± 0.11 | 1.52 ± 0.07 |
| BY173 | 22.29 ± 0.36 | 4.60 ± 0.18 | 2.56 ± 0.09 | 52.13 ± 2.72 | 3.54 ± 0.41 | 17.58 ± 1.52 | 8.04 ± 0.22 | 0.95 ± 0.03 | 1.29 ± 0.04 |
| BY228 | 33.02 ± 0.49 | 4.77 ± 0.21 | 3.27 ± 0.52 | 56.9 ± 4.70 | 2.22 ± 1.93 | 12.66 ± 2.07 | 5.72 ± 1.08 | 1.09 ± 0.11 | 1.14 ± 0.11 |
| BY269 | 23.27 ± 2.41 | 4.29 ± 0.14 | 3.68 ± 0.11 | 54.15 ± 2.01 | 2.15 ± 1.87 | 14.58 ± 0.56 | 8.21 ± 0.52 | 1.21 ± 0.07 | 1.19 ± 0.07 |
| BY241 | 24.92 ± 1.53 | 4.11 ± 0.07 | 2.57 ± 0.12 | 60.21 ± 2.83 | 2.58 ± 2.25 | 12.50 ± 0.30 | 7.41 ± 0.20 | 0.93 ± 0.03 | 1.26 ± 0.01 |
| BY351 | 27.12 ± 1.67 | 4.27 ± 0.09 | 2.08 ± 0.16 | 62.11 ± 0.97 | 0.69 ± 1.20 | 13.27 ± 0.98 | 6.75 ± 0.38 | 0.76 ± 0.07 | 1.19 ± 0.09 |
| BY54 | 30.34 ± 1.91 | 4.24 ± 1.55 | 3.01 ± 0.02 | 52.94 ± 4.64 | 2.33 ± 2.03 | 17.32 ± 0.34 | 6.39 ± 0.19 | 1.10 ± 0.03 | 1.08 ± 0.02 |
| BY223 | 26.14 ± 1.09 | 3.35 ± 1.30 | 2.85 ± 0.23 | 60.82 ± 0.66 | 2.80 ± 0.19 | 12.62 ± 1.14 | 7.22 ± 0.77 | 1.06 ± 0.20 | 1.27 ± 0.04 |
| BY365 | 31.05 ± 1.13 | 4.20 ± 0.18 | 3.52 ± 0.20 | 61.22 ± 3.62 | 2.76 ± 2.65 | 10.08 ± 0.72 | 4.92 ± 0.56 | 1.27 ± 0.05 | 1.13 ± 0.07 |
| BY58 | 35.21 ± 1.51 | 3.95 ± 0.12 | 2.36 ± 0.19 | 57.17 ± 0.79 | 1.66 ± 1.43 | 15.94 ± 0.42 | 7.85 ± 0.26 | 0.92 ± 0.27 | 1.20 ± 0.06 |
| BY371 | 30.5 ± 1.04 | 3.85 ± 0.12 | 2.81 ± 0.15 | 63.63 ± 1.46 | 1.16 ± 1.02 | 10.05 ± 0.53 | 5.94 ± 0.61 | 1.15 ± 0.22 | 1.32 ± 0.08 |

FW, fresh weight;
Each value is the mean result from triplicate ± SD.

### Example 4

### Constructs for Ectopic Expression of Enzymes Involved in Carotenoid Synthesis

### Construct 710-433

A 930 bp ORF fragment of CrtH1 (encoding beta carotene hydroxylase) was amplified by PCR from cDNA prepared from the flower petals of Adonis aestivalis (SEQ ID NO:37; FIG. 10A). The following forward and reverse primers having built-in BamHI and Sad sites, respectively, we used in the amplification: forward,

(SEQ ID NO: 26)

5′-GCGGATCCAATGCTAGCTTCAATGGCAGCGGCA-3′,

and reverse,

(SEQ ID NO: 27)

5′-GCGAGCTCCTATAAGGCATTCATACGCTTTATTCTTC-3′.

Alignment of the amino acid sequences of beta-carotene hydroxylases of various organisms revealed two highly conserved amino acid motifs: VGAAVGME and AHQLHHTDK (FIG. 10B). The cDNA clone of 1,187 by representing CrtH1 encoded a predicted protein of 309 amino acids with a molecular weight of >>35 kDa and a pI of 9.15.

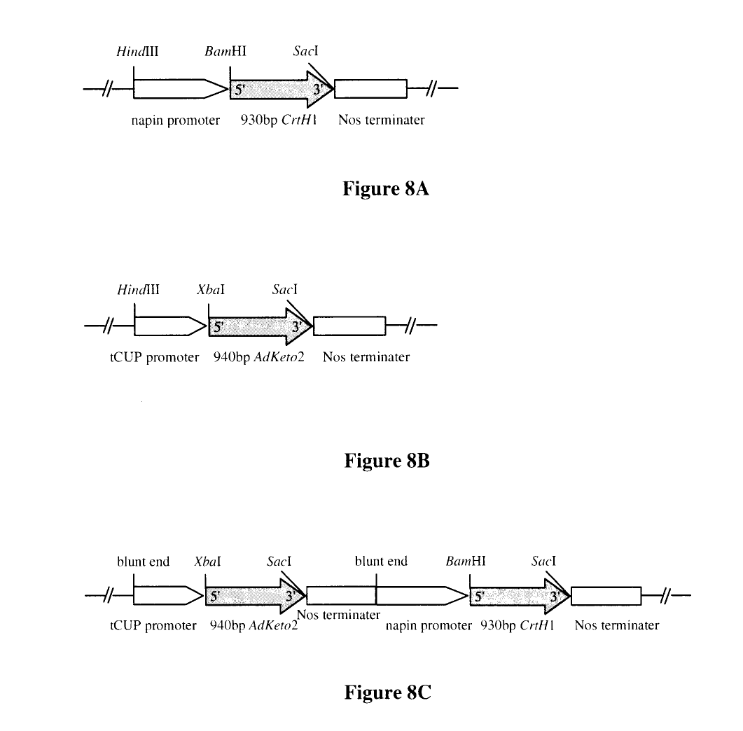

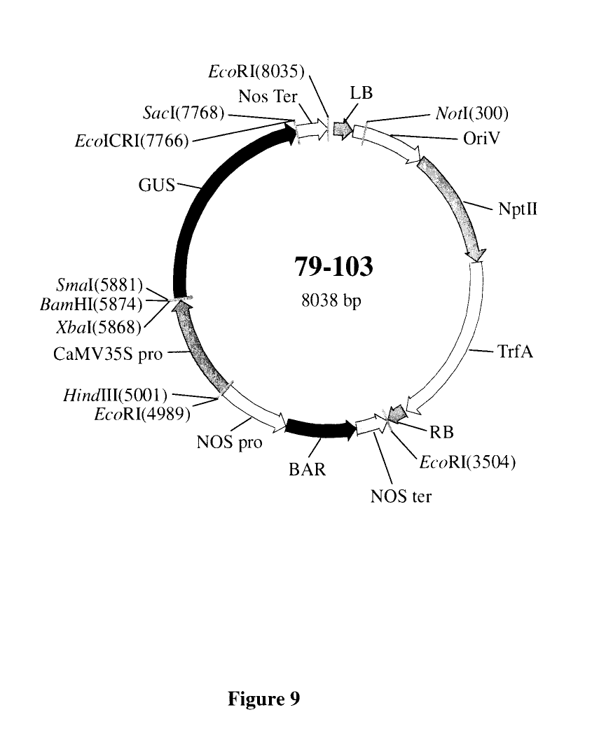

The PCR product was digested with BamHI and Sad and ligated between the BamHI and Sad sites of pBluescriptII KS (+) vector, in which the napin promoter of Brassica napus was cloned between the HindIII and BamHI sites. A ˜2.1 kbp fusion fragment of the napin promoter and the ORF of CrtH1 was then excised by digestion with HindIII and Sad, and cloned between the HindIII and Sad sites of vector, p79-103 (FIG. 9), harbouring a BAR gene for glyphosinate selection in plants. The 710-433 construct is shown in FIG. 8a.

This construct is introduced into wild type B. napus (DH12075), and B. napus lines that express lycopene epsilon cyclase RNAi, for example but not limited to B173, BY269, BY365, as outlined above, and the carotenoid content of these plants is analyzed as outlined above. This construct was also introduced into wild type Arabidopsis thaliana, and β-hydroxylase 1/β-hydroxylase 2 (b1 b2) double-mutant background, in which both Arabidopsis β-carotene hydroxylases are disrupted.

### Construct 710-438

A 940 bp ORF fragment of Adketo2 (encoding beta carotene 3-hydroxylase) was amplified by PCR from cDNA prepared from the flower petals of Adonis aestivalis (SEQ ID NO:38). The following forward and reverse primers having built-in XbaI and Sad sites, respectively, were used: forward,

SEQ ID NO: 29

5′-GCTCTAGAGATGGCAGCAGCAATTTCAGTGTTCA-3′,

and reverse,

SEQ ID NO: 30

5′-GCGAGCTCTCAGGTAGATGGTTGCGTTCGTTTAGT-3′..

The PCR product was digested with XbaI and Sad and ligated between the XbaI and Sad sites of pBluescriptII KS (+) vector, in which the tCUP promoter of tobacco (WO99/67389, which is incorporated herein by reference) was cloned between the HindIII and XbaI sites. This construct was named as 710-437. A ˜1.6 kbp fusion fragment of the tCUP promoter and the ORF of Adketo2 was then excised from construct 710-437 by digestion with HindIII and Sad, and cloned between the Hindu and Sad sites of an in house-built vector, p79-103, harbouring a BAR gene for glyphosinate selection in plants. The 710-438 construct is shown in FIG. 8b.

This construct is introduced into wild type B. napus (DH12075), and B. napus lines that express lycopene epsilon cyclase RNAi, for example but not limited to B173, BY269, BY365, as outlined above, and the carotenoid contact of these plants is analyzed as outlined above.

### Construct 710-440

A 1.6 kbp fusion fragment of the tCUP promoter and the ORF of Adketo2 was excised from construct 710-437 by digestion with HindIII and Sad, and cloned between the HindIII and Sad sites of pBI121. This construct was named as 710-439. A fragment of 710-439 cut with HindIII and EcoRI was filled-in with klenow and blunt-end ligated into HindIII site (klenow filled-in) of 710-433. The construct, 710-440, comprising both Adketo2 and CrtH1 is shown in FIG. 8c.

This construct is introduced into wild type B. napus (DH12075), and B. napus lines that express lycopene epsilon cyclase RNAi, for example but not limited to B173, BY269, BY365, as outlined above, and the carotenoid contact of these plants is analyzed as outlined above.

### Analysis

Expression of 710-433 (crtH1) in Arabidopsis thaliana, wild type and double b1b2 mutant plants, was confirmed by Northern analysis using a 350 by fragment from the 5′-end of the CrtH1 ORF (FIG. 10D). Northern-blot analysis was conducted on total RNA isolated from immature siliques of select transgenic lines. The transgenic lines had significant levels of CrtH1 expression, whereas no expression could be detected in untransformed control plants. No obvious visible phenotypic differences were observed among A. thaliana wt, b1b2 mutant and transgenic lines expressing CrtH1 (data not shown).

Forty four transgenic lines of B. napus were obtained for plants expressing β-carotene hydroxylase (710-433), 33 lines for plants expressing β-carotene ketolase (710-438), and 38 lines for plants expressing both β-carotene hydroxylase and β-carotene ketolase (710-440). Carotenoid analysis on the seeds from these transgenic lines was determined using the method of Yu et al. (2007, Planta, DOI 10.1007/s11248-007-9131-x).

Approximately 200 mg of seed in 3 ml extraction solvent (hexane/acetone/ethanol, 50/25/25) were pulverized by rapidly shaking for 30 min in a scintillation vial containing a steel rod (adapted from Shewmaker et al. 1999). The sample was centrifuged for 10 minutes at 1,800 g and the supernatant collected. The pellet was washed with another 3 ml extraction solvent and the supernatant collected and pooled. The solvent was removed by evaporation at room temperature under a stream of nitrogen gas. Triacyl glycerides were saponified in the residue by heating at 80° C. for 1 hour in 5 ml methanolic-KOH (10% w/v KOH in methanol:water [80:20 v/v]). Carotenoids and aqueous compounds were partitioned using 2 ml H₂O and 3 ml petroleum ether. The ether phase and two 3 ml ether washes were collected, pooled and the solvent evaporated at room temperature under a nitrogen gas stream. The residue was resuspended in 200 μl of acetonitrile/methylene chloride/methanol (50/40/10 [v/v]) with 0.5% (w/v) butylated hydroxytoluene and filtered through a 0.2 μm pore size nylon syringe filter into an HPLC sample vial.

The extract is immediately analysed using HPLC. Aliquots of 20 μl were loaded onto a 4.6 μm×250 mm reverse-phase C₃₀ YMC “Carotenoid Column” (Waters Ltd, Mississauga, ON, Canada) at 35° C. Mobile phases consisted of methanol (A) and tert-methyl butyl ether (B). A linear gradient starting at 95% A and 5% B, proceeding to 35% A and 65% B over 25 min and a flow rate of 1.2 ml·min⁻¹ is used for elution. Compounds in the eluate were monitored at 450 nm using a photodiode array. Peaks are identified by their retention time and absorption spectra compared to those of known standards (CaroteNature, Switzerland). Quantification of carotenoids is conducted using curves constructed with authentic standards.

Mature seeds from A. thaliana were assessed for the presence and levels of carotenoids. HPLC traces of representative wt and b1b2 mutant lines expressing CrtH1, as well as those of wild type and b1b2 mutant are illustrated in FIG. 10E, which shows the heterologous expression of CrtH1 in A. thaliana caused an increase in the level of violaxanthin (peak 1).

Seeds from transgenic A. thaliana plants, both wild type and the double mutant b1b2 A. thaliana, containing A. aestivus crtH1, contained less β-carotene and more violaxanthin, lutein and, in some lines, cryptoxanthin than non-transformed controls (Table 6). As violaxanthin results from the epoxidation of zeathanthin, the at least three-fold greater concentration of violaxanthin in the transgenic lines, compared with untransformed lines, indicated that the crtH1 enzyme hydroxylated β-carotene to zeaxanthin, which was converted to violaxanthin by endogenous zeaxanthin epoxidase.

Decreases in the range of 15-78% in the amount of beta-carotene were observed in most of the transgenic lines compared to untransformed plants; this is expected as a result of enhanced hydroxylation and conversion of beta-carotene to xanthophylls. Expression of CrtH1 in the wild type line resulted in 44-64% increase in lutein content, except for the BY275 and BY284 lines, which did not show any significant difference in lutein level. With the exception of the BY330 line, all b1b2 mutant lines expressing CrtH1 showed increases in lutein levels ranging from 14 to 72% as compared to b1b2 mutant. The hydroxylation of the beta-ring of beat-carotene is required for its conversion to lutein. Therefore, enhanced biosynthesis of lutein in lines expressing CrtH1 would be expected if this gene encodes a beat-carotene hydroxylase.

TABLE 6
Concentrations of carotenoids in Arabidopsis thailana seeds from select
lines transformed with crtH1 gene.

| | violaxanthin | lutein μg/g | zeaxanthin | β-cryptoxanthin | β-carotene |
|---|---|---|---|---|---|
| | μg/g dw | dw | μg/g dw | μg/g dw | μg/g dw |
| wt | 0.694 ± 0.056 | 13.380 ± 0.729 | 0.955 ± 0.051 | UD | 1.890 ± 0.504 |
| wt + crtH1 | | | | | |
| BY275 | 3.186 ± 0.544 | 13.413 ± 3.354 | 0.776 ± 0.193 | UD | 1.009 ± 0.188 |
| BY280 | 5.47 ± 0.342 | 21.631 ± 0.941 | 1.162 ± 0.036 | UD | 0.422 ± 0.033 |
| BY281 | 6.162 ± 0.416 | 20.684 ± 0.623 | 1.067 ± 0.033 | UD | 0.4 ± 0.007 |
| BY284 | 2.939 ± 0.199 | 12.678 ± 1.367 | 0.642 ± 0.100 | UD | 0.718 ± 0.081 |
| BY287 | 7.572 ± 0.284 | 22.016 ± 0.483 | 1.077 ± 0.011 | UD | 0.591 ± 0.068 |
| BY293 | 5.611 ± 0.216 | 21.49 ± 0.597 | 0.895 ± 0.034 | 0.098 ± 0.002 | 1.491 ± 0.142 |
| BY294 | 3.975 ± 0.12 | 19.306 ± 0.95 | 0.883 ± 0.016 | UD | 1.015 ± 0.074 |
| b1b2 mutant | 0.881 ± 0.036 | 26.214 ± 2.690 | 0.711 ± 0.082 | 0.153 ± 0.014 | 8.238 ± 1.019 |
| b1b2 mutant + | | | | | |
| crtH1 | | | | | |
| BY317 | 24.741 ± 2.37 | 42.523 ± 2.275 | 0.727 ± 0.023 | UD | 2.473 ± 0.439 |
| BY330 | 5.522 ± 1.295 | 25.912 ± 4.857 | 0.544 ± 0.048 | 0.139 ± 0.043 | 6.41 ± 0.081 |
| BY334 | 8.713 ± 1.092 | 34.809 ± 2.493 | 0.611 ± 0.039 | 0.156 ± 0.006 | 8.942 ± 0.88 |
| BY341 | 11.005 ± 0.548 | 33.705 ± 2.331 | 0.526 ± 0.061 | 0.216 ± 0.075 | 5.78 ± 0.435 |
| BY342 | 20.62 ± 0.688 | 45.199 ± 0.709 | 0.731 ± 0.025 | UD | 3.099 ± 0.228 |
| BY346 | 15.111 ± 1.103 | 36.464 ± 1.591 | 0.584 ± 0.037 | 0.164 ± 0.013 | 3.262 ± 0.11 |
| BY347 | 3.871 ± 0.163 | 30.08 ± 1.882 | 0.616 ± 0.014 | 0.148 ± 0.015 | 6.985 ± 0.515 |

UD, undetectable;
dw, dry weight;
Each value is the mean result from triplicate ± SD

Characterization of B. napus Lines Expressing crtH1 (p710-433)

Extracts obtained from seeds of eight wild type transgenic lines showed an overall increase in the levels of different carotenoids, especially β-carotene and lutein (Table 7).

TABLE 7
Carotenoid concentrations in transgenic Brassica seeds with construct
p710-433;

| | Violaxanthin | Lutein | Zeaxanthin | Astaxanthin | β-carotene |
|---|---|---|---|---|---|
| DH12075 | UD | 0.69 ± 0.27 | UD | UD | 0.31 ± 0.23 |
| 179 | 0.26 ± 0.07 | 6.34 ± 1.28 | 0.22 ± 0.05 | UD | 3.75 ± 0.91 |
| 181 | UD | 2.89 ± 1.07 | UD | UD | 0.85 ± 0.30 |
| 184 | UD | 1.62 ± 0.23 | UD | UD | 0.74 ± 0.31 |
| E1298 | UD | 2.81 ± 0.13 | UD | UD | 1.07 ± 0.15 |
| E1305 | 0.18 ± 0.03 | 3.05 ± 0.52 | 0.14 ± 0.01 | UD | 2.18 ± 0.77 |
| E1307 | 0.08 ± 0.03 | 1.16 ± 0.03 | UD | UD | 0.20 ± 0.02 |
| E1315 | UD | 1.61 ± 0.24 | UD | UD | 0.18 ± 0.05 |
| E1321 | UD | 1.14 ± 0.05 | UD | UD | 0.06 ± 0.01 |
| E1374 | UD | 1.67 | UD | UD | 1.05 |
| E1343 | UD | 3.86 | UD | UD | 2.77 |
| E1299 | UD | 3.38 | 0.09 | UD | 0.67 |
| E1309 | 0.17 | 7.95 | 0.28 | UD | 0.46 |
| N01-429 | UD | 1.26 ± 0.26 | UD | UD | 0.14 ± 0.02 |
| E1522 | 0.09 ± 0.04 | 2.52 ± 0.51 | 0.09 ± 0.04 | UD | 2.20 ± 0.38 |
| E1536 | UD | 1.77 ± 0.56 | UD | UD | 1.40 ± 0.90 |
| E1640 | 0 | 1.58 ± 0.23 | UD | UD | 0.78 ± 0.10 |
| E1527 | 0.18 ± 0.09 | 5.82 ± 0.37 | UD | UD | 0.78 ± 0.13 |
| E1523 | 0.04 | 4.21 | UD | UD | 0.62 |
| E1518 | 0.57 | 9.33 | UD | UD | 0.98 |
| E1638 | UD | 2.32 | UD | UD | 1.47 |
| E1644 | 0.12 | 2.55 | 0.13 | UD | 1.79 |

Values expressed as μg/g FW;
UD, undetectable;
FW, fresh weight

Characterization of B. napus Lines Expressing adketo2 (p710-438)

Extracts obtained from wild type seeds from at least seven transgenic lines showed significant increases in the levels of β-carotene and lutein (Table 8). In lines derived from the B. napus DH12075.

TABLE 8
Carotenoid concentrations in transgenic Brassica seeds with construct
p710-438

| Plant line | Violaxanthin | Lutein | Zeaxanthin | Astaxanthin | β-carotene |
|---|---|---|---|---|---|
| DH12075 | UD | 0.69 ± 0.27 | UD | UD | 0.31 ± 0.23 |
| DE1368 | UD | 2.37 ± 0.28 | UD | 2.01 ± 0.15 | 1.82 ± 0.833 |
| DE1369 | UD | 1.21 ± 0.15 | UD | 1.72 ± 0.01 | 0.71 ± 0.107 |
| DE1371 | UD | 1.90 ± 0.64 | UD | 1.96 ± 0.09 | 1.64 ± 0.38 |
| DE1372 | UD | 1.40 ± 0.37 | UD | 1.93 ± 0.10 | 0.47 ± 0.21 |
| DE1178 | UD | 3.43 | 0.33 | 2.32 | 1.92 |
| DE1200 | 0.15 | 9.11 | UD | 2.31 | 2.62 |
| DE1210 | UD | 2.28 | UD | 1.83 | 3.49 |
| DE1216 | 0.25 | 11.09 | 1.65 | 2.97 | 4.74 |
| DE1318 | UD | 1.12 | UD | UD | 0.73 |
| DE1364 | 0.09 | 2.47 | UD | 2.26 | 7.09 |
| DE1373 | UD | 3.75 | 0.15 | 2.7 | 4.07 |
| YN01- | UD | 1.26 ± 0.26 | UD | UD | 0.14 ± 0.02 |
| 429 | | | | | |
| DE1551 | UD | 2.15 ± 0.31 | UD | UD | 1.02 ± 0.15 |
| DE1660 | UD | 2.36 ± 0.45 | UD | UD | 0.47 ± 0.07 |
| DE1541 | 0.09 | 6.17 | UD | 1.83 | UD |
| DE1544 | UD | 1.35 | UD | UD | UD |
| DE1554 | UD | 3.28 | 0.16 | 1.64 | UD |
| DE1558 | 0.09 | 3.72 | UD | 1.86 | UD |

Values expressed as μg/g FW;
UD, undetectable;
FW, fresh weight

Characterization of B. napus Lines Expressing crtH1 and adketo2 (p710-440)

Extracts obtained from wild type seeds from ten transgenic plants showed increased levels of β-carotene and lutein, and six had astaxanthin (FIG. 10E, Table 9). Transgenic lines derived from both parental lines, DH12075 and YN01-429, (a yellow seeded B. napus), had astaxanthin in the seed at about 2 μg/g FW.

TABLE 9
Carotenoid concentrations in transgenic Brassica seeds with construct
p710-440

| Plant line | Violaxanthin | Lutein | Zeaxanthin | Astaxanthin | β-carotene |
|---|---|---|---|---|---|
| DH12075 | UD | 0.69 ± 0.27 | UD | UD | 0.31 ± 0.23 |
| DE1212 | UD | 2.87 | UD | 1.87 | 4.96 |
| DE1236 | UD | 4.81 | UD | UD | 0.72 |
| DE1247 | UD | 5.13 | UD | 2.11 | 1.48 |
| DE1323 | UD | 4.12 | UD | 1.81 | 0.55 |
| DE1339 | UD | 3.00 | UD | 2.46 | 2.41 |
| DE1340 | UD | 2.84 | UD | UD | 0.75 |
| YN01-429 | UD | 1.26 ± 0.26 | UD | UD | 0.14 ± 0.02 |
| DE1493 | 0.07 ± 0.03 | 2.13 ± 0.33 | UD | UD | 1.09 ± 0.47 |
| DE1495 | UD | 3.50 ± 1.22 | UD | UD | 0.22 ± 0.10 |
| DE1499 | 0.21 ± 0.01 | 5.53 ± 0.21 | UD | UD | 0.87 ± 0.065 |
| DE1664 | UD | 11.48 ± 2.01 | UD | UD | 0.97 ± 0.62 |
| DE1671 | 0.65 ± 0.09 | 6.52 ± 0.29 | UD | 1.96 ± 0.05 | 0.79 ± 0.09 |
| DE1675 | 0.17 ± 0.01 | 5.07 ± 0.81 | 0.09 ± 0.01 | 1.77 ± 0.02 | 6.59 ± 0.88 |
| DE1677 | 0.09 ± 0.04 | 3.91 ± 0.27 | UD | UD | 0.57 ± 0.03 |
| DE1672 | 0.22 | 6.86 | 0.103 | UD | UD |
| DE1678 | 1.33 | 17.65 | 0.214 | 5.41 | UD |

Values expressed as μg/g FW;
UD, undetectable;
FW, fresh weight

The above results demonstrate that expression of crtH1, adketo2, or both crtH1 and adketo2 resulted in production of functional enzyme in A. thaliana and B. napus germplasm, and that seeds with elevated levels of astaxanthin and beta-carotene may be produced.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

Bartley G E, Scolnik P A (1995) Plant carotenoid: pigments for photoprotection, visual attraction, and human health. Plant Cell 7:1027-1038
Bartley G E, Scolnik, P. A., Beyer P., (1999) Two Arabidopsis thaliana carotene desaturases, phytoene desaturase and ζ-carotene desaturase, expressed in Escherichia coli, catalyze a poly-cis pathway to yield pro-lycopene Eur. J. Biochem. 259:396-403
Botella-Pavia P, Rodríguez-Concepción M (2006) Carotenoid biotechnology in plants for nutritionally improved foods. Physiol. Plant. 126:369-381
Church G, Gilbert W (1984) Genome Sequencing. Proc Natl Aca Sci USA 81:1991-1995
Cunningham F X Jr, Gantt E (1998) Genes and enzymes of carotenoid biosynthesis in plants. Annu. Rev. Plant Physiol. Plant Mol. Biol. 49:557-583
Demmig-Adams B and Adams III W W (2002) Antioxidants in photosynthesis and human nutrition. Science 298:2149-2153
Ducreux L J M, Morris W L, Hedley P E, Shepherd T, Davies H V, Millam S, Taylor M A (2005). Metabolic engineering of high carotenoid potato tubers containing enhanced levels of β-carotene and lutein. J. Exp Bot 56:81-89
Fire A, Xu S, Montgomery M K, Kostas S A, Driver S E, Mello C C (1998) Potent and specific genetic interference by double-stranded RNA in Caenorhabditis elegans. Nature 391:806-811
Fraser P D and Bramley P M (2004) The biosynthesis and nutritional uses of carotenoids. Prog Lipid Res 43:228-265
Giuliano G, Bartley G E, Scolnik P A (1993) Regulation of carotenoid biosynthesis during tomato development. Plant Cell 5:379-387
Horiguchi G (2004) RNA silencing in plants: a shortcut to functional analysis. Differentiation 72: 75-73
Howitt C A, Pogson B J (2006) Carotenoid accumulation and function in seeds and non-green tissues. Plant Cell and Environment 29:435-445
Krinsky N I and Johnson E J (2005) Carotenoid actions and their relation to health and disease. Mol Aspects Med. 26459-516
Landrum J T and Bone R A (2004) Dietary lutein and zeaxanthin: reducing the risk of macular degeneration. Agro Food Industry Hi-Tech 15:22-25
Lakshman M R and Okoh C (1993) Enzymatic conversion of all trans-beta-carotene to retinal. Meth Enzymol 214:256-269
Li L, Lu S, Cosman K M, Earle E D, Garvin D F, O'Neill J (2006) β-Carotene accumulation induced by cauliflower Or gene is not due to an increase capacity of biosynthesis. Phytochemistry 67:1177-1184
Latowski D, Banas A K, Strzalka K, Gabrys H. (2007) Amino sugars—new inhibitors of zeaxanthin epoxidase, a violaxanthin cycle enzyme. J Plant Physiol. 2007 Mar. 7; 164:231-237 (Epub 2006 Oct. 30)
Lindgren L, Stahlberg K G, Hoglund A S (2003) Seed-specific overexpression of an endogenous Arabidopsis phytoene synthase gene results in delayed germination and increased levels of carotenoids, chlorophyll, and abscisic acid. Plant Physiol 132:779-785
Misawa N, Truesdale M R, Sandmann G. Fraser P D. Bird C. Schuch W. Bramley P M., (1994) Expression of a Tomato cDNA Coding for Phytoene
Synthase in Escherichia coli, Phytoene Formation In Vivo and In Vitro, and Functional Analysis of the Various Truncated Gene Products, J. Biochem, Vol. 116:980-985
Moloney M M, Walker J M, Sharma K K (1989) high efficiency transformation of Brassica napus using Agrobacterium vectors. Plant Cell Report 8: 238-242
Peter G F, Thornber J P (1991) Biochemical composition and organization of higher plant photosystem II light-harvesting pigment-protein. J Biol Chem 266:16745-16754
Pogson B J, McDonald K, Truong M, Britton G, DellaPenna D (1996) Arabidopsis carotenoid mutants demonstrate lutein is not essential for photosynthesis in higher plants. Plant Cell 8:1627-1639
Rosati C, Aquilani R, Dharmapuri S, Pallara P, Marusic C, Tavazza R, Bouvier F, Camara B, Giuliano G (2000) Metabolic engineering of beta-carotene and lycopene content in tomato fruit. Plant J 24: 413-419
Shewmaker C K, Sheey J A, Daley M, Colburn S, Ke D Y (1999) Seed-specific overexpression of phytoene synthase: increase in carotenoids and other metabolic effects. Plant J 20:401-412
Stringham, G R (1971) Genetics of four hypocotyl mutants in Brassica campestris L. J. Hered. 62: 248-250
Taylor M and Ramsay G (2005) Carotenoid biosynthesis in plant storage organs: recent advances and prospects for improving plant food quality. Physiol Plant 124:143-151
Wesley S V, Helliwell C A, Smith N A, Wang M, Rouse D T, Liu Q, Gooding P S, Singh S P, Abbott D, Stoutjesdijk P A, Robinson S P, Gleave A P, Green A G, Waterhouse P M (2001) Construct design for efficient, effective and high-throughout gene silencing in plants. Plant J. 27:581-590
Ye X, Al-Babili S, Kloti A, Zhang J, Lucca P, Beyer p, Potrykus I (2000) Engineering the provitamin A (β-carotene) biosynthetic pathway into (carotenoid free) rice endosperm. Science 287:303-305
Young, L W, Jalink, H, Denkert, R and Reaney M T J (2006) Factors affecting the density of Brassica napus seeds. Seed Science and Technology 34:633-645
Yu. B. Lydate D J. Schafer U A. Hannoufa A., (2006) Planta 14:

What is claimed is:

1. A method for altering the level of one or more carotenoid in a plant or a tissue within the plant comprising,
i) introducing a nucleic acid sequence into the plant, the nucleic acid sequence comprising a regulatory region operatively associated with a silencing nucleotide sequence, wherein expression of the silencing nucleotide sequence reduces or eliminates the expression of a lycopene epsilon cyclase, and
ii) expressing the silencing nucleotide sequence within the plant or a tissue within the plant to reduce the level of the lycopene epsilon cyclase in the plant or within a tissue of the plant, thereby altering the level of the one or more carotenoid in the plant or plant tissue,
the reduced level of lycopene epsilon cyclase determined by comparing the level of expression of the lycopene epsilon cyclase in the plant, or a tissue of the plant, with a level of lycopene epsilon cyclase in a second plant, or the tissue from the second plant, that does not express the silencing nucleic acid sequence.

2. The method of claim 1, wherein the silencing nucleotide sequence is selected from the group consisting of an antisense RNA encoding nucleotide sequence, a ribozyme encoding sequence, and an RNAi encoding nucleotide sequence.

3. The method of claim 1 wherein the regulatory region is selected from the group consisting of a constitutive regulatory region, an inducible regulatory region, a developmentally regulated regulatory region, and a tissue specific regulatory region.

4. The method of claim 3, wherein the regulatory region is a tissue specific regulatory region.

5. The method of claim 1 wherein the level of the one or more than one carotenoid is reduced by about 25 to about 100%, where compared to the level of the same one or more than one carotenoid obtained from second plant.

6. The method of claim 1, wherein, the silencing nucleotide sequence reduces the level of expression of lycopene epsilon cyclase, while the level of expression of lycopene beta cyclase remains similar to that of a second plant, or the tissue from the second plant, that does not express the silencing nucleotide sequence, and the reduced level of lycopene epsilon cyclase determined by comparing the level of expression of the lycopene epsilon cyclase in the plant, or a tissue of the plant, with a level of lycopene epsilon cyclase in the second plant, or the tissue from the second plant, that does not express the silencing nucleic acid sequence.

7. The method of claim 1, wherein the silencing nucleotide sequence is selected from the group consisting of nucleotides 28-384 of SEQ ID NO:4, and 1411-1835 of SEQ ID NO:4, or a nucleotide sequence that hybridizes to nucleotides 28-384 of SEQ ID NO:4, and 1411-1835 of SEQ ID NO:4, or that hybridizes to a complement of nucleotides 28-384 of SEQ ID NO:4, and 1411-1835 of SEQ ID NO:4, under stringent hybridization conditions, the stringent hybridization conditions comprising hybridization in Church buffer at 61° C. for 22 hr, washing the filter twice in 2×SSC, 0.1% SDS for 10 min at 61° C., and washing twice in 0.2×SSC, 0.1% SDS for 10 min at 61° C., wherein the silencing nucleotide sequence exhibits reduces expression of a lycopene epsilon cyclase (ε-CYC) gene or sequence from about 10 to about 100%.

8. A nucleic acid sequence comprising, a regulatory region operatively associated with a silencing nucleotide sequence that reduces or eliminates the expression of a lycopene epsilon cyclase, and does not alter the level of expression of lycopene beta cyclase.

9. The nucleic acid sequence of claim 8, wherein the silencing nucleotide sequence is selected from the group consisting of an antisense RNA encoding nucleotide sequence, a ribozyme encoding sequence, and an RNAi encoding nucleotide sequence.

10. The nucleic acid sequence of claim 9, wherein the regulatory region is selected from the group consisting of a constitutive regulatory region, an inducible regulatory region, a developmentally regulated regulatory region, and a tissue specific regulatory region.

11. The nucleic acid sequence of claim 10, wherein the regulatory region is a tissue specific regulatory region.

12. The nucleic acid sequence of claim 9, wherein the silencing nucleotide sequence is selected from the group consisting of nucleotides 28-384 of SEQ ID NO:4, and 1411-1835 of SEQ ID NO:4, or a nucleotide sequence that hybridizes to nucleotides 28-384 of SEQ ID NO:4, and 1411-1835 of SEQ ID NO:4, or that hybridizes to a complement of nucleotides 28-384 of SEQ ID NO:4, and 1411-1835 of SEQ ID NO:4, under stringent hybridization conditions, the stringent hybridization conditions comprising hybridization in Church buffer at 61° C. for 22 hr, washing the filter twice in 2×SSC, 0.1% SDS for 10 min at 61° C., and washing twice in 0.2×SSC, 0.1% SDS for 10 min at 61° C.

13. A construct comprising the nucleic acid sequence of claim 8.

14. A plant comprising the nucleic acid sequence of claim 8.

15. A seed comprising the nucleic acid sequence of claim 8.

16. A method for altering the level of one or more carotenoid in a plant or a tissue within the plant comprising,
i) introducing a nucleic acid sequence into the plant, the nucleic acid sequence comprising a regulatory region operatively associated with crtH1, adketo2, or a combination of crtH1 and adketo2 and,
ii) expressing the nucleic sequence within the plant or a tissue within the plant thereby altering the level of the one or more carotenoid in the plant or plant tissue.
